(19) United States
(12) Patent Application Publication
(10) Pub. No.: US 2017/0362635 A1
(43) Pub. Date: Dec. 21, 2017

(54) MUSCLE-SPECIFIC CRISPR/CAS9 EDITING OF GENES

(71) Applicant: University of Washington, Seattle, WA (US)

(72) Inventors: Jeffrey S. Chamberlain, Seattle, WA (US); Niclas Bengtsson, Seattle, WA (US); Stephen D. Hauschka, Seattle, WA (US)

(73) Assignee: University of Washington, Seattle, WA (US)

(21) Appl. No.: 15/628,533

(22) Filed: Jun. 20, 2017

Related U.S. Application Data
(60) Provisional application No. 62/352,505, filed on Jun. 20, 2016.

(51) Int. Cl. C12Q 1/68; C07H 21/02; C12N 15/11; C12N 15/63; C12N 9/22; C07H 21/04; A61K 38/43; C12N 15/10; C12N 15/00; C12N 2310/20

(52) U.S. Cl. CPC C12Q 1/68; A61K 38/43; C07H 21/02; C12N 15/111; C12N 15/102; C12N 15/63; C12N 9/22; C07H 21/04; C12N 2310/20; C12N 2310/10; C12N 15/00

(57) ABSTRACT

Pharmaceutical compositions including a muscle-specific nuclease cassette, one or more guide RNA cassettes, and a delivery system for delivery of the muscle-specific nuclease cassette and the one or more gRNA cassettes are provided. The pharmaceutical composition may also include a mutation-corrected DNA template including a modification to be made in a target nucleic acid sequence. Methods for treating a subject having a muscular or neuromuscular disorder are also provided. The methods may include administering to the subject a therapeutically effective amount of the pharmaceutical composition. Methods of modifying or editing the sequence of a target nucleic acid sequence in a muscle cell are also provided. The methods may include contacting or transducing the muscle cell with a muscle-specific nuclease cassette and one or more gRNA cassettes.

### CROSS-REFERENCE TO RELATED APPLICATIONS

This application claims the benefit of U.S. Provisional Application No. 62/352,505, filed Jun. 20, 2016, which is hereby incorporated by reference in its entirety.

### STATEMENT REGARDING FEDERALLY SPONSORED RESEARCH

This invention was made with government support under Grant No. R01 AR044533, awarded by the National Institutes of Health. The government has certain rights in the invention.

### TECHNICAL FIELD

The present disclosure relates generally to pharmaceutical compositions including a muscle-specific nuclease cassette, one or more guide RNA (gRNA) cassettes, and a delivery system for the muscle-specific nuclease cassette and the one or more gRNA cassettes. The pharmaceutic composition may also include a normal or mutation-corrected DNA template carrying a modification to be made in a target nucleic acid sequence (e.g., a homology template). The present disclosure also relates to methods for treating a subject having a muscular or neuromuscular disorder. In particular, the methods may include administering to the subject a therapeutically effective amount of the pharmaceutical composition. The present disclosure also relates to methods of modifying or editing the sequence of a target nucleic acid sequence in a muscle cell and/or a muscle progenitor cell. The methods may include contacting or transducing the muscle cell and/or the muscle progenitor cell with a muscle-specific nuclease cassette and one or more gRNA cassettes. The methods may also include contacting or transducing the muscle cell and/or the muscle progenitor cell with a mutation-corrected DNA template including a modification to be made in the target nucleic acid sequence. The muscle-specific nuclease cassette, the one or more gRNA cassettes, and/or the mutation-corrected DNA template may be present on a single piece of DNA or on two or more pieces of DNA.

### BACKGROUND

A variety of approaches for gene therapy of Duchenne muscular dystrophy (DMD) are in development, including microdystrophin delivery by adeno-associated virus (AAV) vector (see Gregorevic, P. et al., Nature Med. 10, 828-834, doi:10.1038/nm1085 (2004); Bengtsson, N. E., et al. Hum. Mol. Genet., doi:10.1093/hmg/ddv420 (2015); and Chamberlain, J. R., et al. Mol. Ther. 25, 1125-1131, http://dx.doi.org/10.1016/j.ymthe.2017.02.019 (2017)). However, microdystrophins are not fully functional and episomal AAV vectors could be gradually lost during normal myofiber turnover. An emerging, alternative strategy is to modify the dystrophin gene using the CRISPR/Cas9 system, such as has recently been shown by deletion of an exon in the mdx^{ScSn} mouse model of DMD (see Tabebordbar, M., et al. Science, doi:10.1126/science.aad5177 (2015); Nelson, C. E., et al. Science, doi: 10.1126/science. aad5143 (2015); and Long, C., et al. Science, doi:10.1126/science.aad5725 (2015)). However, strategies to apply gene editing to the dystrophin gene will require great flexibility due to its frequency (approximately 1:5000 newborn males) and the high incidence of spontaneous new mutations in this X-linked recessive disorder (see Emery, A. E. H., et al. Duchenne Muscular Dystrophy. 3rd edn, (Oxford University Press, 2003) and Mendell, J. R., et al. Annals of neurology 71, 304-313, doi:10.1002/ana.23528 (2012)). Mutations in the 2.2 megabase dystrophin gene result in loss of expression of both dystrophin and the dystrophin-glycoprotein complex, causing muscle membrane fragility and progressive muscle wasting (see Emery, A. E. H., et al. Duchenne Muscular Dystrophy. 3rd edn, (Oxford University Press, 2003) and Batchelor, C. L., et al. Trends Cell Biol. 16, 198-205, doi:10.1016/j.tcb.2006.02.001 (2006)). AAV vectors derived from serotypes 6, 8, and 9 have shown considerable promise in animal models for DMD by enabling systemic delivery of genetic cassettes that can partially compensate for the absence of dystrophin (see Gregorevic, P., et al. Nature Med. 10, 828-834, doi:10.1038/nm1085 (2004) and Bengtsson, N. E., et al. Hum. Mol. Genet., doi:10.1093/hmg/ddv420 (2015)). While AAVs do not exclusively target striated muscle, highly restricted muscle transduction can be achieved by using muscle-specific gene regulatory cassettes (see Salva, M. Z., et al. Mol. Ther. 15, 320-329, doi:10.1038/sj.mt.6300027 (2007)). An inherent limitation to AAV vector-mediated dystrophin replacement is the inability to fit the 14 kilobase (kb) cDNA into the ˜5 kb vector packaging limit. Microdystrophins that lack non-essential domains dramatically improve muscle pathophysiology in dystrophic animal models, yet do not fully restore muscle strength (see Harper, S. Q., et al. Nature Med. 8, 253-261, doi:10.1038/nm0302-253 (2002); Rahimov, F., et al. J. Cell Biol. 201, 499-510, doi:10.1083/jcb.201212142 (2013); Banks, G. B., et al. PLoS Genet. 6, e1000958, doi:10.1371/journal.pgen.1000958 (2010); and Lai, Y., et al. J. Clin. Invest. 119, 624-635, doi:10.1172/JCI36612 (2009).

### BRIEF DESCRIPTION OF THE DRAWINGS

The embodiments disclosed herein will become more fully apparent from the following description and appended claims, taken in conjunction with the accompanying drawings.

FIG. 1 depicts strategies for creating a dystrophin mRNA carrying an ORF by removing the mdx^{4cv} TAA premature stop codon (the mdx^{4cv} C to T point-mutation is underlined). Panel A depicts strategy 1 (Δ5253), which utilizes both dual- and single-AAV vector approaches to target introns 51 and 53 (arrows=sgRNA target sites shown in a 5′-3′ direction based on target strand) to direct excision of exons 52 and 53 (panel B). Panel C depicts strategy 2 (53*), which utilizes a dual-vector approach to target exon 53 on either side of the stop codon, relying on homology directed repair (HDR) utilizing a WT DNA template, or non-homologous end joining (NHEJ) to generate either full-length WT dystrophin (panel D) or a partial in-frame deletion of exon 53 (panel E).

FIG. 2 depicts that in vivo gene editing can introduce a functional ORF in mdx^{4cv} mouse muscles. Panel A is a graph depicting deep sequencing quantification on PCR amplicons generated from pooled genomic DNA extracted from muscles treated with strategy 1 (Δ5253, n=4), demonstrating successful gene editing at each of the individual target regions. Shown are the percentages of total reads that displayed genomic modifications occurring as a result of NHEJ (including insertions, deletions, and substitutions) at sgRNA target sites in introns 51 and 53. Panel B depicts RT-PCR of target region transcripts isolated from TAs treated with strategy 1 (Δ5253, n=4) showing a predominant shorter product (black box), corresponding to approximately 87.5% of total transcripts based on image densitometry. Panel C depicts subclone sequencing of the treatment-specific RT-PCR product (black box in panel b) confirmed that these transcripts lacked the sequences encoded on exons 52 and 53 (the novel junction between exons 51 and 54 is highlighted by a dashed-line box). Panel D depicts deep sequencing quantification of gene editing efficiency on PCR amplicons generated from pooled genomic DNA (left, n=5) and RT-PCR amplicons generated from pooled transcripts (right, n=4) extracted from muscles treated with strategy 2 (53*). Shown are the percentages of total reads that displayed genomic modifications occurring as a result of NHEJ, HDR, or via a combination of both, at both sgRNA target sites in exon 53. Panel E depicts deep sequencing reading frame analysis for strategy 2 (53*), showing the percentage of total edited transcript (RNA) and genomic (DNA) reads resulting in frameshift indels, in-frame indels, in-frame deletions without the TAA stop codon (pΔ53), HDR reads (not including mixed NHEJ/HDR reads), and the total percentage of edited reads encoding a functional dystrophin ORF (HDR/pΔ53).

FIG. 3 illustrates that dystrophin expression in treated muscles can improve muscle morphology. Panel A is an image depicting TA muscles from treated mice that were collected and analyzed for expression of the mCherry reporter gene (top) or cryosectioned for immunostaining of dystrophin (bottom). Widespread dystrophin expression resulted from both strategies 1 and 2 (Scale bar, 1 mm). Panel B is a western analysis of muscles from treated and untreated mice (WT and mdx^{4cv}) showing dystrophin (Dys), SpCas9, SaCas9, and GAPDH expression. Dystrophin was detected using antisera raised against the C terminus (CT). The SaCas9 nuclease carried an HA epitope tag to enable detection with anti-HA antibodies. Panel C is a graph depicting quantification of GAPDH-normalized dystrophin expression in treated TAs compared with WT muscles (n=4). Panel D is a graph depicting analysis of immunostained cross-sections from treated and control mice for the percentage of all myofibers expressing dystrophin (n=5). Panel E is a graph showing the cross-sectional area (CXA) size distribution of individual myofibers from treated and control muscles (n>12,500 total fibers per group). Panel F is a graph depicting the total myogenic cross-sectional area (CXA) that was dystrophin-positive for treated and WT control muscles (n=5). Panel G is an array of charts depicting individual myofiber size distribution for treated TAs relative to dystrophin expression. Panel H is a graph depicting the percentage of myofibers containing centrally located nuclei for dystrophin-positive treated myofibers and for total myofibers of control TA muscles (n=5). Data are shown as mean±s.e.m. ***P<0.001, (One-way ANOVA multiple comparisons test with Turkey's post hoc test).

FIG. 4 illustrates that CRISPR/Cas9-mediated dystrophin correction localizes nNOS to the sarcolemma and can improve muscle function. Panel A illustrates immunofluorescent (IF) staining for nNOS, laminin, and dystrophin in IM-treated and control muscles (Scale bar, 100 μm). Panel B is a graph depicting specific force generating levels of treated mdx^{4cv} mouse TA muscles 18 weeks post-IM transduction with 2.5×10¹⁰ vector genomes (v.g.) of each AAV vector (SaCas9 Δ5253 (n=8), SpCas9/Δ5253 (n=6), SpCas9/53* (n=8), and of untreated age-matched WT (n=3) and mdx^{4cv} (n=6) muscles. Bars represent mean±s.e.m. (*P<0.05, ***P<0.001). Panel C is a graph depicting protection against eccentric contraction-induced injury as demonstrated by measuring contractile performance immediately before increasing length changes during maximal force production in TA muscles of untreated (n=5) versus IM-treated mdx^{4cv} mice (SaCas9Δ5253 (n=8), SpCas9/Δ5253 (n=7), SpCas9/53*(n=8)). Values are represented as mean±s.e.m. Statistical significance was determined via multiple Student's t-test comparisons, (**P<0.01, ****P<0.0001).

FIG. 5 illustrates that systemic gene editing can result in widespread dystrophin expression. Immunofluorescence analysis of mdx^{4cv} mouse muscles at 4 weeks post systemic transduction with dual (sp5253) and single (sa5253) vector approaches in strategy 1 is shown. Panel A is a muscle cross-section showing widespread transduction of multiple muscle groups following high dose (1×10¹³/4×10¹² v.g. of nuclease/targeting vectors) dual-vector delivery based on mCherry reporter gene expression, Scale bar, 3 mm. Whole cardiac cross-sections showing dystrophin expression following dual-vector delivery at the high dose (panel B), low dose (panel C, 1×10¹²/1×10¹²) and following single vector delivery at the low dose (panel D, 1×10¹²), Scale bars, 1 mm. Insets depict magnified fields of view. Widespread but variable dystrophin expression is observed in multiple muscle groups following high dose dual-vector delivery; including TA (panel E), diaphragm (panel F), soleus (panel G), and gastrocnemius (panel H), Scale bars, 100 μm. Western analysis of cardiac lysates demonstrates expression of near full-length dystrophin in low dose (LD) and high dose (HD) treatment groups, with increased dystrophin expression at higher vector doses (panel I).

FIG. 6 illustrates in vitro validation of targeting constructs. In vitro editing efficiency at target sites within exon 53 (53*, strategy 2) as well as within introns 51 and 53 (Δ5253, strategy 1) was determined via the T7 endonuclease 1 assay following nuclease- and targeting-construct electroporation into primary dermal fibroblasts isolated from mdx^{4cv} mice. For strategy 2, the two target sites within exon 53 were analyzed together due to their close proximity to each other. Efficiency estimated via densitometry measurements of unique cleavage bands.

FIG. 7 illustrates analyses of gene editing efficiency for strategy 1. Graphs generated by the CRISPRESSO™ software pipeline during genomic deep sequencing analysis of PCR amplicons generated across the target sites within intron 51 (i51) and intron 53 (i53) for strategy 1 (Δ5253). Shown in panel A are the percentages of insertions, deletions, and substitutions, resulting from NHEJ events, for each nucleotide position across the PCR amplicon (left panels). The y-axis represents % total genomes or (% genomes, number of genomes exhibiting NHEJ). Also shown is the average insertion size (center panels) and average deletion size (right panels) at each nucleotide position across the PCR amplicons. Dotted lines represent predicted Cas9 cleavage sites. PCR across the ˜45 kb region targeted for deletion on DNA isolated from TAs treated with strategy 1 (Δ5253, n=4) in panel B shows the presence of a unique product (arrow) that by subsequent cloning and sequencing was determined to correlate to a merging of introns 51 and 53 as predicted.

FIG. 8 illustrates analyses of gene editing efficiency for strategy 2. Graphs generated during CRISPRESSO™ analysis for strategy 2 (53) during deep sequencing analysis of pooled PCR (panel A, n=5), and RT-PCR (panel B, n=4) amplicons generated across the target sites within exon 53 for strategy 2 (53*). Shown are the percentages of insertions, deletions, and substitutions, resulting from NHEJ events, for each nucleotide position across the amplicons (left panels). The y-axis represents % total genomes or (% genomes, number of genomes exhibiting NHEJ). Also shown is the average insertion size (center panels) and average deletion size (right panels) at each nucleotide position across the amplicons. Dotted lines represent predicted Cas9 cleavage sites. In panel C, RT-PCR of target region transcripts isolated from TAs treated with strategy 2 (53*, n=4) shows the presence of a shorter product (lower black box) making up approximately 20% of total transcripts (based on image densitometry). Subsequent cloning and sequencing revealed that this product corresponded to out-of-frame transcripts lacking the sequences encoded on exon 53. In panel D, T7 endonuclease 1 digestion of the predominant top RT-PCR product from muscles treated with strategy 2 (upper black box in panel C) indicates the presence of unique transcripts, making up approximately 11.2% of the analyzed RT-PCR product based on image densitometry (top; arrows). Also shown is the DNA sequence of one clone of the RT-PCR products, revealing an in-frame transcript where the nonsense mutation was removed by a 27 bp in-frame deletion (bottom).

FIG. 9 illustrates HDR and reading frame analyses for strategy 2. Graphical representation of HDR detection, reading frame analysis, and distribution of HDR genotypes for exon 53 based on deep sequencing of pooled PCR amplicons generated from genomic DNA (top, n=5) or transcripts (cDNA) (bottom, n=4) isolated from muscles treated with strategy 2 (53*). “Mutation position distribution of HDR” panels: Shown are the percentages of HDR-derived nucleotide substitutions for each position across the amplicons, as generated by the CRISPRESSO™ software pipeline. The y-axis represents % total genomes or (% genomes, number of genomes exhibiting HDR). The graph demonstrates nucleotide substitutions at sites corresponding to the two silent PAM site mutations (G to A) and at the site of the mdx^{4cv} C to T point mutation. Dotted lines represent predicted Cas9 cleavage sites. “Frameshift profile” and “In-frame profile” panels: Shown are the size distributions of frameshift and in-frame reads, as generated by the CRISPRESSO™. The y-axis represents % of frameshift or in-frame reads while the x-axis represent the size of the corresponding deletions, insertions, and substitutions. “DNA” and “RNA” panels: Shown are the genotypes and corresponding frequencies of HDR events resulting in the substitution of the mdx^{4cv} T mutation to the WT C nucleotide. Genotypes resulting from successful HDR consisted of substitutions for: the complete HDR template (HDR), a partial HDR template from the 5′ or 3′ ends (5′/3′-pHDR), and substitution of T to C without the PAM site mutations (WT). The silent PAM site mutations are depicted in bold. Of note, WT genotypes may in fact contain a large proportion of background reads, based on the level of reads observed with random single nucleotide substitutions and in untreated control RNA samples (see FIGS. 15 and 16). The remaining HDR reads containing 2 or 3 defined HDR specific nucleotide substitutions appear highly specific as these reads exhibit close to zero prevalence in untreated RNA samples (see FIG. 16).

FIG. 10 is an image depicting immunofluorescent analysis for the single vector approach in strategy 1. Dystrophin expression detected in treated TA muscles injected with the single AAV6/SaCas9Δ5253 vector (n=4), analyzed at 4 weeks post-transduction (scale bar=1000 μm).

FIG. 11 illustrates the size distribution analysis for individual myofibers in treated TA muscles. Cross-sectional area of individual, dystrophin-positive and dystrophin-negative myofibers from transduced TA muscles (n=4 (Δ5253), n=5 (53*) muscles; >25,000 myofibers traced per treatment). Transduced myofibers expressing dystrophin were larger than degenerating dystrophin-negative myofibers following IM injection (panels A and B), with a 5- and 10-fold reduction in myofibers under 250 μm², respectively.

FIG. 12 depicts data on varying the ratio of Cas9 versus gRNA vectors when delivered intravascularly to dystrophic mice, as well as adjusting the dose of vectors. The upper panels show dystrophin expression in the heart (as detected by immunofluorescence) after systemic delivery of the indicated doses of each AAV6 vector. For simplicity, a dose of 1×10¹³ v.g. is abbreviated as 1E13. The lower panels also relate to the issue of muscle-specificity, as they show no gene editing in liver (lower left panel) and no Cas9 expression in liver (lower right panel). In the lower left panel, PCR was used to estimate the amount of each vector present in nucleic acids extracted from target tissues (upper portion of panel), or the amount of correctly edited dystrophin mRNA present in nucleic acids extracted from target tissues (lower portion of panel). The lower right panel depicts a western blot used to detect dystrophin or Cas9 expression in control hearts, or in hearts and livers of vector treated mdx^{4cv} mice.

FIG. 13 is an overview of the HDR strategy. In this example, two silent mutations were introduced into the HDR template to prevent vector cleavage by Cas9 and to facilitate distinguishing gene correction events generated via HDR from incompletely corrected HDR events or background mutations in the mdx^{4cv} muscle DNA or RNA.

FIG. 14 is an analysis of gene editing efficiency and successful HDR at 4 weeks post-treatment.

FIG. 15 is a table depicting manual genotype analysis of genomic DNA for strategy 2. Manual analysis of deep sequencing reads within the Fastq file for on-target PCR amplicons generated from DNA isolated from muscles treated according to strategy 2 (53*). Top: prevalence of deep sequencing reads corresponding to different HDR derived genotypes and random single nucleotide substitutions (proposed background) at sites of particular interest. Bottom: prevalence of selected sequences corresponding to partial in-frame deletions (pΔ53) resulting in the removal of the mdx^{4cv} stop codon. The 28 bp out-of-frame deletion sequence predicted to be most prevalent, resulting from DNA cleavage at the prototypical PAM-3 nucleotide position, is indeed found most frequently among the deletions followed by a 27 bp in-frame deletion sequence.

FIG. 16 is a table depicting manual genotype analysis of transcripts for strategy 2. Manual analysis of deep sequencing reads within the Fastq files for on-target RT-PCR amplicons generated from RNA isolated from untreated (mdx control) and treated muscles according to strategy 2 (53*). Top: prevalence of deep sequencing reads corresponding to different HDR derived genotypes and random single nucleotide substitutions (proposed background) at sites of particular interest. The presence of reads with single nucleotide substitutions within the untreated sample indicates the level of natural variation and/or sequencing errors. Inclusion of 2 or more substitutions at defined positions in the query virtually eliminates detection in untreated controls. Bottom: prevalence of selected sequences corresponding to partial in-frame deletions (pΔ53) resulting in the removal of the mdx^{4cv} stop codon. The sequence generated following removal of 27 bp between the two target sites appears to be the most prevalent. The 28 bp out-of-frame deletion sequence, predicted to be most prevalent following cleavage at the prototypical PAM-3 nucleotide position, appears less prominent at the transcript level then at the DNA level (as shown in FIG. 15).

FIG. 17 is a table depicting genomic off-target analyses. Deep sequencing quantification of gene editing frequency at the top predicted potential off-target sites for each gRNA reveals low levels of sequence variation (nucleotide mismatches from the on-target sequence are in bold). The vast majority of edited reads detected at potential off-target sites correspond to single nucleotide substitutions. Few deletion and insertion events are randomly distributed across the amplicons, indicating low levels of natural variation and/or sequencing errors.

FIG. 18 is the sequence of the HDR fragment used in the AAV vectors shown in FIG. 1, panel C and FIG. 13. The silent PAM site mutations that were introduced to prevent vector cleavage by Cas9 (G to A) are double underlined. The wild type nucleotide (C) used for replacement of the mdx^{4cv} mutation (T) is single underlined.

FIG. 19 is a list of primers. Primer pairs used for subcloning; SpCas9 from pSpCas9(BB)-2A-Puro (PX459) (ADDGENE™ plasmid# 48139) into the pAAV-CK8-SpCas9 nuclease vector, the U6-(Sp)sgRNA cassette from plasmid lentiCRISPRv1 (ADDGENE™ plasmid #49535) into the pAAV-Δ5253/53* targeting vectors, an additional U6-(Sa)sgRNA cassette into the plasmid pX601-AAV-CMV::NLS-SaCas9-NLS-3xHA-bGHpA;U6::Bsal-sgRNA (ADDGENE™ plasmid #61591), as well as sgRNA target sequences and primers used to amplify ON/OFF target sites for PCR, RT-PCR, and deep sequencing (DS) analyses (sequences are shown in the 5′-3′ orientation).

### DETAILED DESCRIPTION

A pharmaceutical composition may include a muscle-specific nuclease cassette, one or more gRNA cassettes, and a delivery system for delivery of the muscle-specific nuclease cassette and the one or more gRNA cassettes. The pharmaceutical composition may also include a template sequence homologous to a target sequence (e.g., a homology template). Methods for treating a subject having a muscular or neuromuscular disorder may include administering to the subject a therapeutically effective amount of the pharmaceutical composition. Furthermore, methods of modifying or editing the sequence of a target nucleic acid sequence in a muscle cell may include contacting or transducing the muscle cell with a muscle-specific nuclease cassette, one or more gRNA cassettes, and/or a template sequence homologous to a target sequence. The one or more gRNA cassettes may encode a gRNA coding sequence and a mutation-corrected DNA template including a modification to be made in the target nucleic acid sequence. The muscle-specific nuclease cassette, the one or more gRNA cassettes, and/or the template sequence homologous to a target sequence may be carried or delivered in the same vector or in two or more separate vectors.

It will be readily understood that the embodiments, as generally described herein, are exemplary. The following more detailed description of various embodiments is not intended to limit the scope of the present disclosure, but is merely representative of various embodiments. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified.

Unless specifically defined otherwise, the technical terms, as used herein, have their normal meaning as understood in the art. The following terms are specifically defined with examples for the sake of clarity.

As used herein, “a” and “an” denote one or more, unless specifically noted.

As used herein, “about” refers to a quantity, level, value, number, frequency, percentage, dimension, size, amount, weight, or length that varies by as much as about 30%, about 25%, about 20%, about 15%, about 10%, about 9%, about 8%, about 7%, about 6%, about 5%, about 4%, about 3%, about 2%, or about 1% to a reference quantity, level, value, number, frequency, percentage, dimension, size, amount, weight, or length. In any embodiment discussed in the context of a numerical value used in conjunction with the term “about,” it is specifically contemplated that the term “about” can be omitted.

As used herein, an “increased” or “enhanced” amount is typically a “statistically significant” amount, and may include an increase that is about 1.1, about 1.2, about 1.3, about 1.4, about 1.5, about 1.6, about 1.7, about 1.8, about 1.9, about 2, about 2.5, about 3, about 3.5, about 4, about 4.5, about 5, about 6, about 7, about 8, about 9, about 10, about 15, about 20, about 30, about 40, about 50, or more times (e.g., about 100, about 500, about 1,000 times; including all integers and decimal points in between and above 1, e.g., 2.1, 2.2, 2.3, 2.4, etc.) an amount or level described herein. Similarly, as used herein, a “decreased,” “reduced,” or “lesser” amount is typically a “statistically significant” amount, and may include a decrease that is about 1.1, about 1.2, about 1.3, about 1.4, about 1.5, about 1.6 about 1.7, about 1.8, about 1.9, about 2, about 2.5, about 3, about 3.5, about 4, about 4.5, about 5, about 6, about 7, about 8, about 9, about 10, about 15, about 20, about 30, about 40, about 50, or more times (e.g., about 100, about 500, about 1,000 times; including all integers and decimal points in between and above 1, e.g., 3.6, 3.7. 3.8, 3.9, etc.) an amount or level described herein.

As used herein, a “subject” includes any animal that exhibits a disease or symptom, or is at risk for exhibiting a disease or symptom. Suitable subjects include laboratory animals (e.g., mice, rats, rabbits, and guinea pigs), farm animals, and domestic animals or pets (e.g., cats or dogs). Non-human primates and human patients are also included.

As used herein, a “therapeutically effective amount” or a “therapeutically effective dose” refers to an amount of a compound or pharmaceutical composition that, when administered to a subject, is sufficient to effect partial or complete treatment of a disease or condition in the subject. The amount of a compound or pharmaceutical composition that constitutes a “therapeutically effective amount” will vary depending on the compound or pharmaceutical composition, the condition and its severity, the manner of administration, and/or the age of the subject to be treated, but can be determined routinely by one of ordinary skill in the art having regard to his or her own knowledge and to this disclosure. Accordingly, when a compound or pharmaceutical composition is said to possess “therapeutic efficacy,” this is intended to mean that the compound or pharmaceutical composition is capable of effecting treatment of a disease or condition in a subject, provided a “therapeutically effective amount” of the compound or pharmaceutical composition is administered under appropriate conditions.

As used herein, “treating” or “treatment” refers to the treatment of a disease or condition of interest in a subject (e.g., a human) having the disease or condition of interest, and includes: (i) preventing or inhibiting the disease or condition from occurring in the subject, for example, when the subject is predisposed to the condition but has not yet been diagnosed as having the condition; (ii) inhibiting the disease or condition, i.e., arresting its development; (iii) relieving the disease or condition, i.e., causing regression of the disease or condition; and/or (iv) relieving the symptoms resulting from the disease or condition.

As used herein, “disease,” “disorder,” and “condition” may be used interchangeably or may be different in that the particular malady, injury, or condition may not have a known causative agent (so that etiology has not yet been determined), and it is, therefore, not yet recognized as an injury or disease but only as a condition or a syndrome (e.g., an undesirable condition or syndrome), wherein a more or less specific set of symptoms has been identified by clinicians.

The formulations can be prepared in pharmaceutically acceptable, physiologically acceptable, and/or pharmaceutical-grade solutions for administration to a cell or a subject (e.g., an animal), either alone, or in combination with one or more other modalities of therapy. The formulations may be administered in combination with other agents, such as other proteins, polypeptides, pharmaceutically active agents, etc.

The compositions can be administered via any suitable route, including but not limited to, locally, orally, subcutaneously, systemically, intravenously, intravascularly, intramuscularly, mucosally, transdermally (e.g., via a patch), or via a bolus. Accordingly, in addition to these general routes of administration, in some embodiments, the composition may be administered via a mode selected from the group consisting of, but not limited to: parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intratumoral, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intravaginal, buccal, sublingual, and intranasal, and via administration to the central nervous system. The compositions may be encapsulated in liposomes, exosomes, microparticles, microcapsules, nanoparticles, and the like. Techniques for formulating and administering therapeutically useful polypeptides are also disclosed in Remington: The Science and Practice of Pharmacy (Alfonso R. Gennaro, et al. eds. Philadelphia College of Pharmacy and Science 2000), which is incorporated herein in its entirety.

In some embodiments, the compositions of the present disclosure may be administered via a schedule including continuous administration or intermittent administration. Accordingly, in addition to these general schedules, in some embodiments, the composition may be administered twice a day, once a day, once every other day, once a week, once a month, or another suitable period of administration.

Microdystrophins, which lack non-essential domains, are not fully functional and in some cases do not fully restore muscle strength. This deficit may be overcome in some mutational contexts (e.g., in the case of small mutations such as point mutations or small deletions that do not remove essential dystrophin gene exons) using the CRISPR/Cas9 system to modify or correct the mutated dystrophin gene in vivo. The potential of this system has previously been demonstrated in patient-derived iPSCs and murine germline manipulation studies (see Li, H. L., et al. Stem Cell Rep., doi:10.1016/j.stemcr.2014.10.013 (2014) and Long, C., et al. Science 345, 1184-1188, doi:10.1126/science.1254445 (2014)). Studies have also utilized the CRISPR/Cas9 system for in vivo excision of dystrophin exon 23 (see Tabebordbar, M., et al. Science, doi:10.1126/science.aad5177 (2015); Nelson, C. E., et al. Science, doi: 10.1126/science. aad5143 (2015); and Long, C., et al. Science, doi:10.1126/science.aad5725 (2015)), which carries a nonsense mutation in the mdx^{ScSn} mouse [see Sicinski, P., et al. Science 244, 1578-1580 (1989)]. As DMD is a new mutation syndrome and more than 4,000 independent mutations have been identified (see http://www_dmd_nl), it has been explored whether alternative gene editing strategies may be developed for more complex mutational contexts. The present disclosure utilized the mdx^{4cv} mouse model that harbors a nonsense mutation within exon 53 (see Im, W. B., et al. Hum. Mol. Genet. 5, 1149-1153 (1996)). Notably, this is homologous to human exon 53 which is within a mutational hot spot region that carries the genetic lesion in ˜60% of patients with DMD-causing deletions (see Flanigan, K. M., et al. Human mutation 30, 1657-1666, doi: 10.1002/humu.21114 (2009)). The mdx^{4cv} model exhibits fewer dystrophin-revertant myofibers than the original mdx^{ScSn} strain and a slightly more progressive phenotype, thus providing a more representative model of DMD. In contrast to exon 23, excision of exon 53 will not restore an open-reading frame (ORF) to the resulting mRNA, therefore a much larger genomic region containing both exons 52 and 53 must be removed or the mutation itself directly targeted. Editing different regions of the vast dystrophin gene could generate very different results as the effects on pre-mRNA splicing and the stability and/or functional properties of modified dystrophins are generally not always predictable (see Harper, S. Q., et al. Nature Med. 8, 253-261, doi:10.1038/nm0302-253 (2002)).

Gene replacement therapies utilizing adeno-associated viral (AAV) vectors hold promise for treating Duchenne muscular dystrophy (DMD). A potentially longer-lasting approach revolves around efforts to directly modify the dystrophin gene using the CRISPR/Cas9 system. Here multiple approaches are provided for editing the mutation in the mdx^{4cv} mouse model for DMD using both single- and dual-AAV vector delivery of a muscle-specific Cas9 cassette together with single-guide RNA cassettes and, in one approach, a dystrophin homology region. Muscle-restricted Cas9 expression was able to lead to direct gene editing of the mutation, multi-exon deletion or complete gene correction via homologous recombination in post-mitotic myofibers. Treated muscles demonstrated production of near- to full-length dystrophin in up to 70% of the myogenic cross-sectional area and a significant increase in force generation.

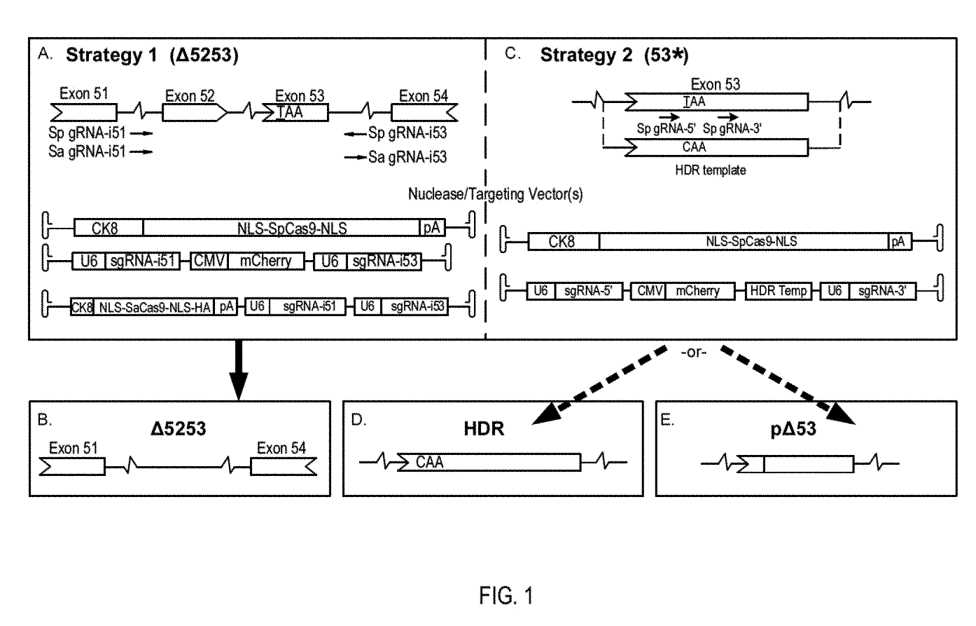

Induction of dystrophin expression was tested following AAV6-mediated delivery of CRISPR/Cas9 components derived from either Streptococcus pyogenes (SpCas9) (see Cong, L., et al. Science 339, 819-823 (2013)) or Staphylococcus aureus (SaCas9) (see Ran, F. A., et al. Nature 520, 186-191 (2015)) using dual- or single-vector approaches, respectively (see FIG. 1, panels A-E). Cas9 expression was restricted to skeletal and cardiac muscle by use of the muscle-specific CK8 regulatory cassette (RC) (see Himeda, C. L., et al. Methods Mol. Biol. 709, 3-19 (2011)) to reduce the risk of off-target events in non-muscle cells and to minimize elicitation of an immune response (see Hartigan-O'Connor, D., et al. Mol. Ther. 4, 525-533 (2001) and Hu, C., et al. Mol. Ther. 22, 1792-1802 (2014)). Several approaches were tested to either excise exons 52 and 53 (Δ5253; strategy 1) or to directly target the mutation in exon 53 (53*; strategy 2). Due to the ˜5 kb packaging limit of AAV, dual-AAV vectors were designed to work in tandem: a nuclease vector expressing SpCas9 under control of the CK8 RC and a set of targeting vectors containing two single-guide RNA (sgRNA) expression cassettes unique to strategies 1 or 2 (see FIG. 1, panels A-E). A variant of strategy 1 relying on CK8-regulated expression of the smaller SaCas9 enabled use of a single vector (see FIG. 1, panel A).

The overall approaches used in strategy 1 (Δ5253) are potentially applicable to a majority of DMD patients with mutations affecting one or more exons whose removal via editing would allow production of an mRNA with an ORF. For this, sgRNAs were designed to direct Cas9-mediated DNA cleavage within the introns flanking exons 52-53 (see FIG. 1, panel A). Following DNA repair via NHEJ these would result in deletion of ˜45 kb of genomic DNA and 330 bp in the encoded mRNA. Successful deletion with strategy 1 can remove the nonsense mutation and lead to the expression of a dystrophin lacking 110 amino acids in a non-essential portion of the protein (see FIG. 1, panel B). Strategy 2 (53*) was developed to target small mutations directly, in this case in exon 53, using two distinct methods. These approaches could be applicable to patients with mutations in exons encoding essential domains of dystrophin, such as the dystroglycan-binding domain (see Abmayr, S., et al. in Molecular Mechanisms of Muscular Dystrophies (ed. Winder S. J. Landes Bioscience (2006)). The first approach within strategy 2 relies on the introduction of a “mutation-corrected” DNA template to allow for potential HDR following Cas9-mediated DNA cleavage, resulting in full-length endogenous dystrophin expression (see FIG. 1, panels C and D). In the absence of successful HDR, this approach could still enable dystrophin expression where NHEJ repair of the cleaved exon 53 leads to excision of the nonsense mutation while maintaining an ORF in the resultant mRNA (see FIG. 1, panels C and E).

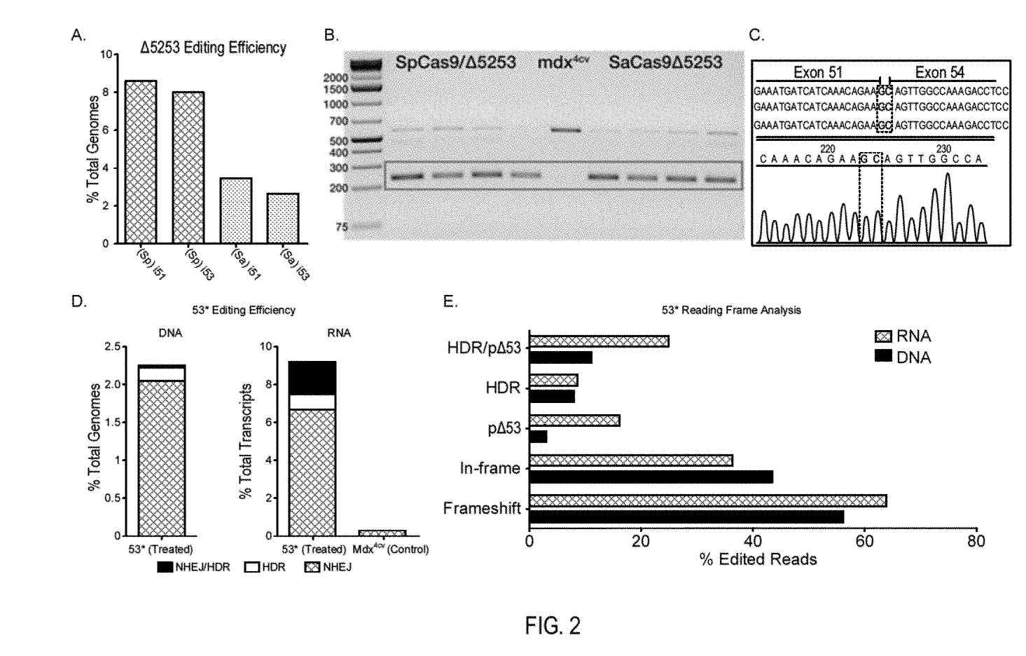

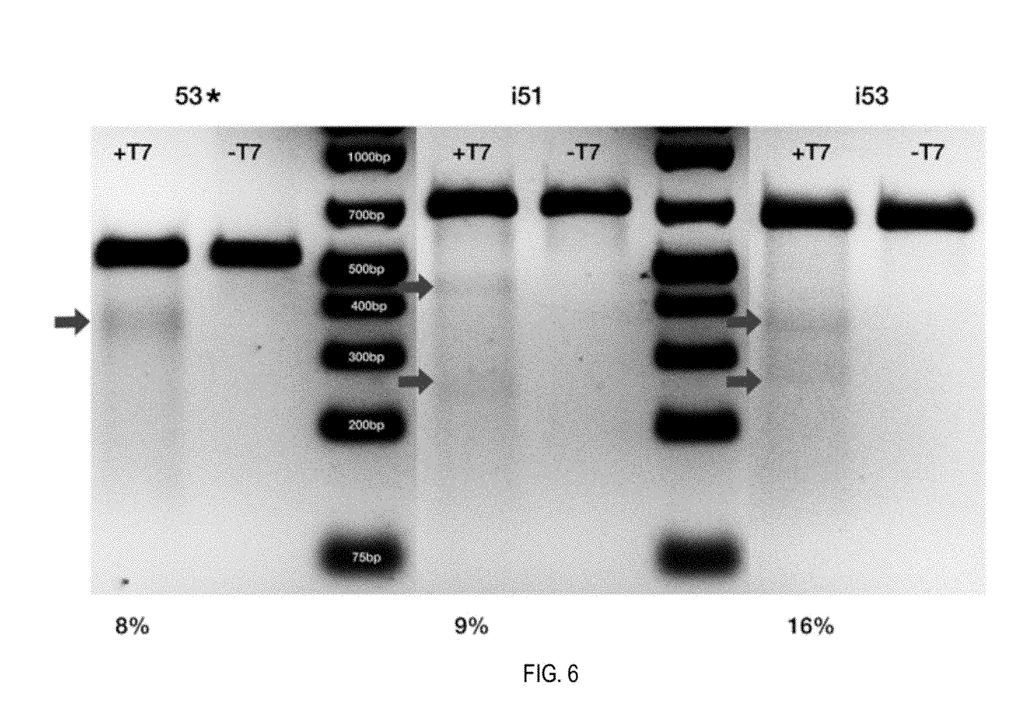

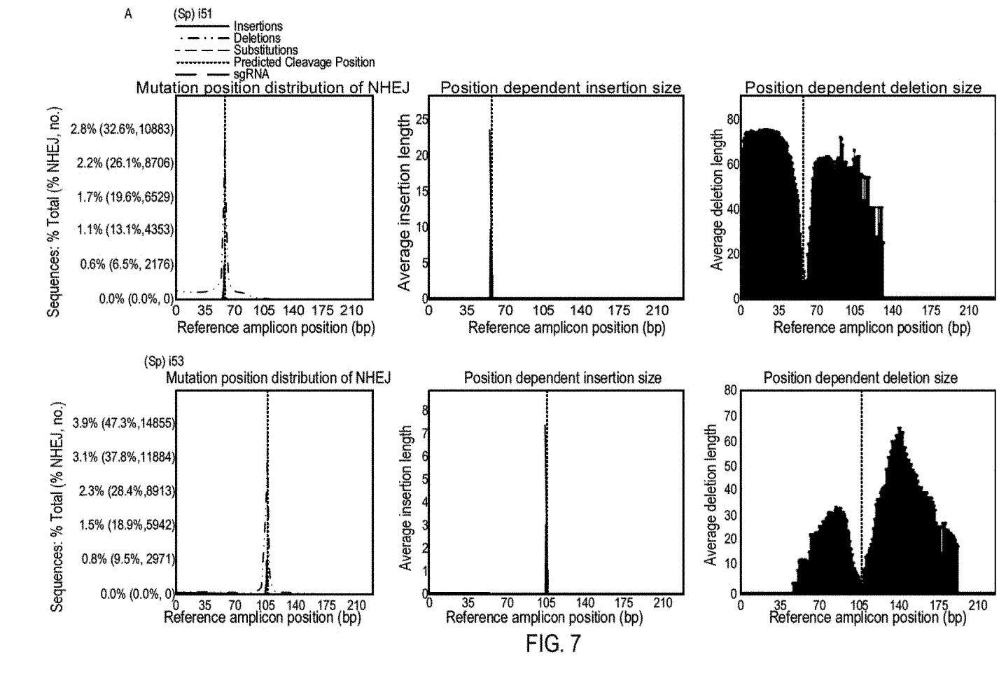

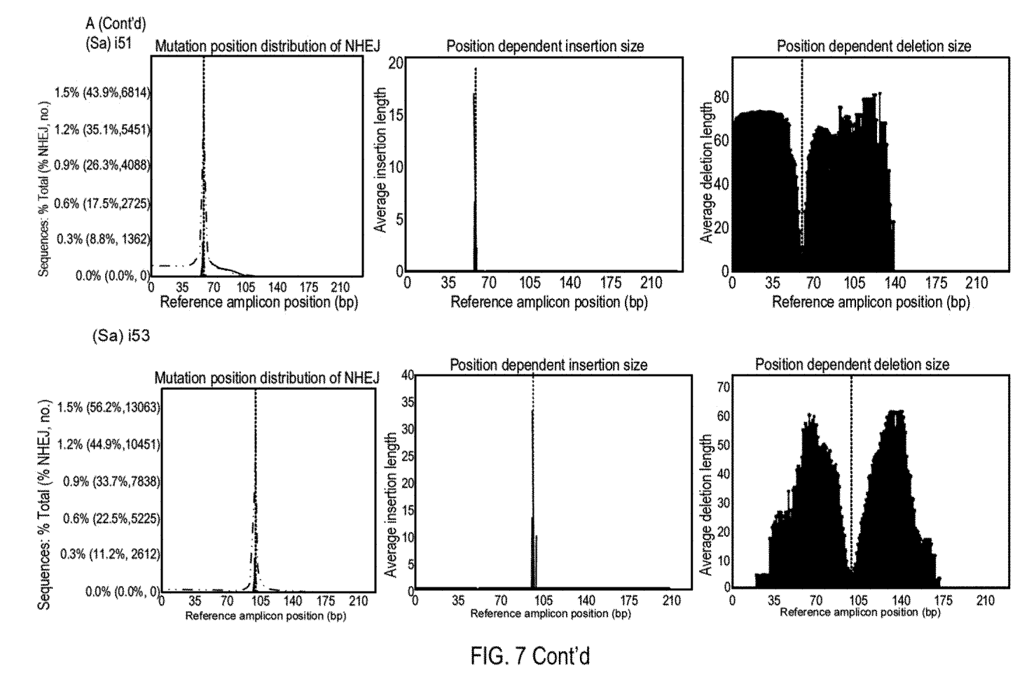

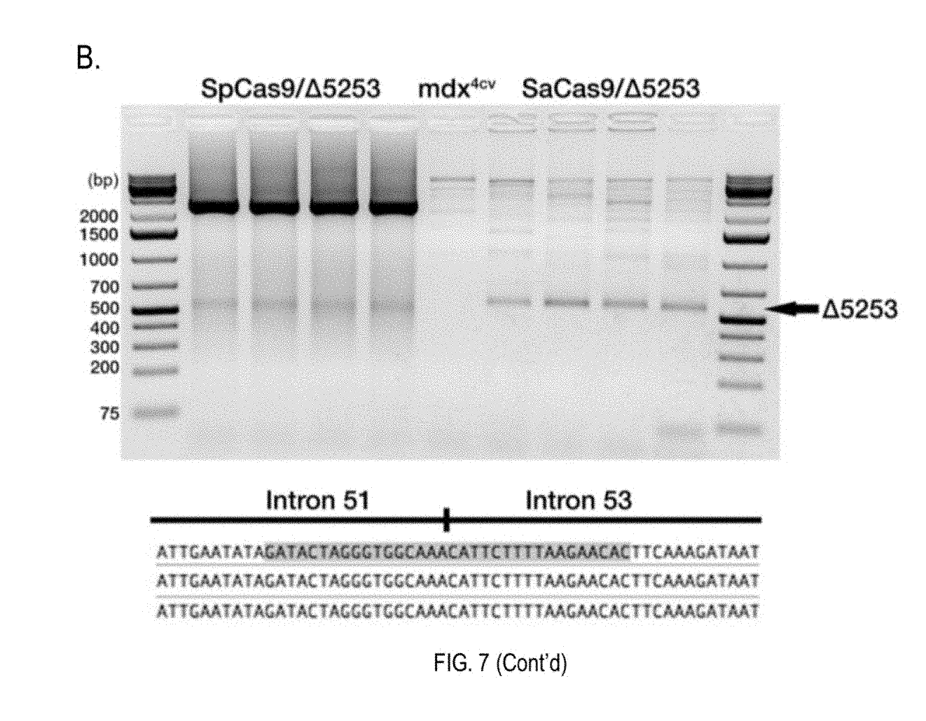

Dystrophin gene targeting was initially evaluated in vitro using the T7 endonuclease 1 assay in mdx^{4cv}-derived primary dermal fibroblasts. The respective targeting efficiencies for sgRNA-i51 and sgRNA-i53 were 9 and 16%, while a combined targeting efficiency of 8% was observed for the 5′ and 3′ sgRNAs within exon 53 (which due to their close proximity were analyzed together (see FIG. 6). For initial in vivo testing, 10-12 week old male mdx^{4cv} mice were injected in the tibialis anterior (TA) muscles with 5×10¹⁰ vector genomes (v.g.) of the AAV6 CK8-nuclease plus targeting vectors and sacrificed at 4 weeks post-injection. In vivo targeting efficiency was estimated via deep sequencing across target regions within the dystrophin gene. For strategy 1 PCR amplification of the genomic DNA region spanning the intron 51-53 target sites revealed low levels of a unique Δ5253 deletion product whose sequence was verified following isolation and cloning (see FIG. 7, panels A and B). Due to the large size of the genomic deletion, quantification of NHEJ events resulting from the deletion of both exons 52 and 53 could not be determined via deep sequencing. However, deep sequencing of PCR amplicons generated across the individual target sites could be used to quantify the instances where on-target DNA cleavage did not result in the excision of the intervening 45 kb segment. Using this approach, gene editing efficiencies at introns 51 and 53, respectively, were 8.6% and 8.2% for the dual-vector (Sp) approach and 3.5% and 2.7% for the single vector (Sa) approach (see FIG. 2, panel A; FIG. 7, panels A and B; and Table 1). Reverse transcription PCR (RT-PCR) analysis revealed a predominant shorter dystrophin transcript that lacked the sequences encoded on exons 52 and 53 as determined by sequencing of the excised unique band (see FIG. 2, panels B and C).

TABLE 1

| ON Target: | sgRNA sequence (5′-3′) | Chromosome | Position |
|---|---|---|---|
| (Sp) i51 | GATACTAGGGTGGCAAATAG | X | 84530675- |
| | (SEQ ID NO: 1) | | 84530694 |
| (Sp) i53 | GTGTTCTTAAAAGAATGGTG | X | 84576353- |
| | (SEQ ID NO: 2) | | 84576372 |
| (Sa) i51 | GATACTAGGGTGGCAAATAGA | X | 84530675- |
| | (SEQ ID NO: 3) | | 84530695 |
| (Sa) i53 | GAGATAAATCCCTGCTTATCAC | X | 84576316- |
| | (SEQ ID NO: 4) | | 84576337 |
| (Sp) 53*-5′ | (G)TCAAGAACAGCTGCAGAAC | X | 84575591- |
| (combined w. 3′) | (SEQ ID NO: 5) | | 84575609 |
| (Sp) 53*-3′ | (G)CAGTTGAATGAAATGTTAA | X | 84575619- |
| (combined w. 5′) | (SEQ ID NO: 6) | | 84595637 |
| (Sp) 53* (Treated, | | | |
| combined) | | | |
| (Sp) 53* (Control, | | | |
| combined) | | | |

| | Total | | | NHEJ/HDR | Editing | HDR |
|---|---|---|---|---|---|---|
| ON Target: | reads | NHEJ | HDR | (mixed) | efficiency (%) | (%) |
| (Sp) i51 | 387126 | 33348 | 0 | 0 | 8.61 | 0.00 |
| (Sp) i53 | 383505 | 31411 | 0 | 0 | 8.19 | 0.00 |
| (Sa) i51 | 448016 | 15533 | 0 | 0 | 3.47 | 0.00 |
| (Sa) i53 | 870140 | 23263 | 0 | 0 | 2.67 | 0.00 |
| (Sp) 53*-5′ | 4681379 | 96177 | 8507 | 1421 | 2.27 | 0.18 |
| (combined w. 3′) | | | | | | |
| (Sp) 53*-3′ | | | | | | |
| (combined w. 5′) | | | | | | |
| (Sp) 53* (Treated, | 336095 | 22245 | 2692 | 5944 | 9.19 | 0.80 |
| combined) | | | | | | |
| (Sp) 53* (Control, | 486042 | 1292 | 0 | 26 | 0.27 | 0.00 |
| combined) | | | | | | |

Table 1 depicts deep sequencing quantification of editing efficiency and HDR events using CRISPRESSO™. Efficient targeting was observed at all target sites for the different approaches. For NHEJ events, the majority of edited reads corresponded to deletions followed by insertions and substitutions (see FIGS. 6 and 7). For strategy 2, on-target deep sequencing was performed on DNA and cDNA generated from RNA isolated from treated muscles. Comparing DNA to RNA revealed an increase in both editing efficiency and prevalence of reads corresponding to successful HDR events at the transcript level, likely due to protection of functional edited transcripts against nonsense mediated decay. The sequence used to detect HDR events using CRISPRESSO™ included the WT cytosine at the site of the point mutation and both PAM site mutations. HDR quantification does not include mixed NHEJ/HDR events.

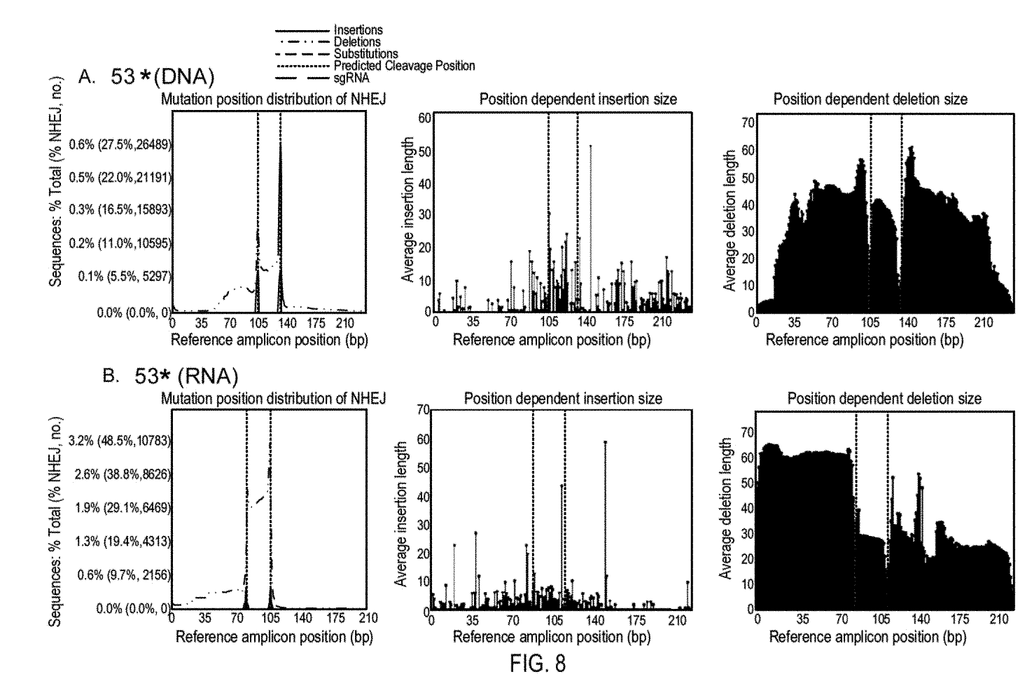

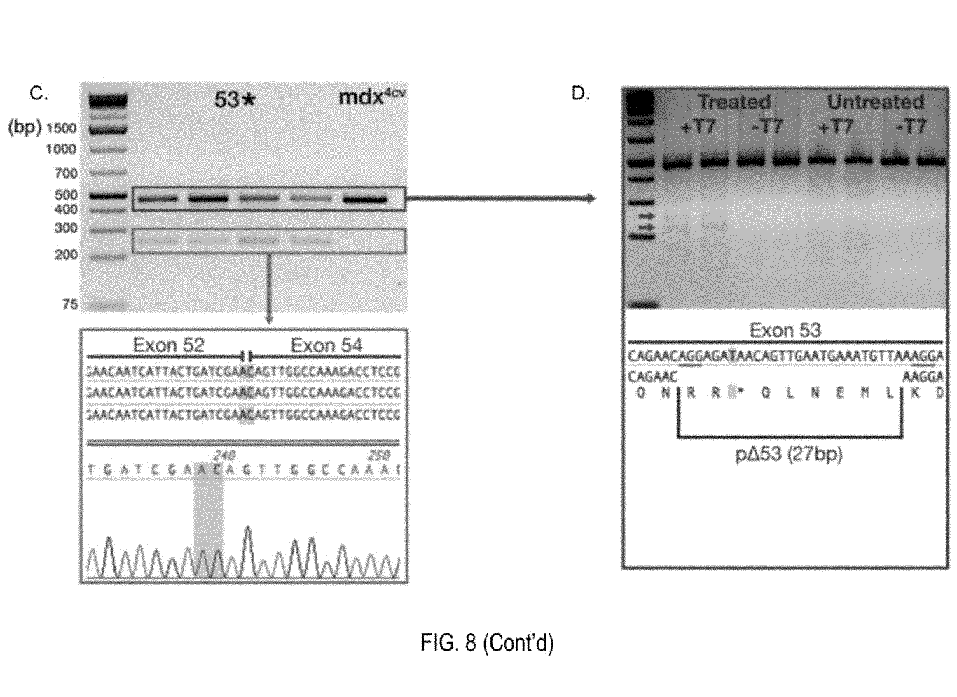

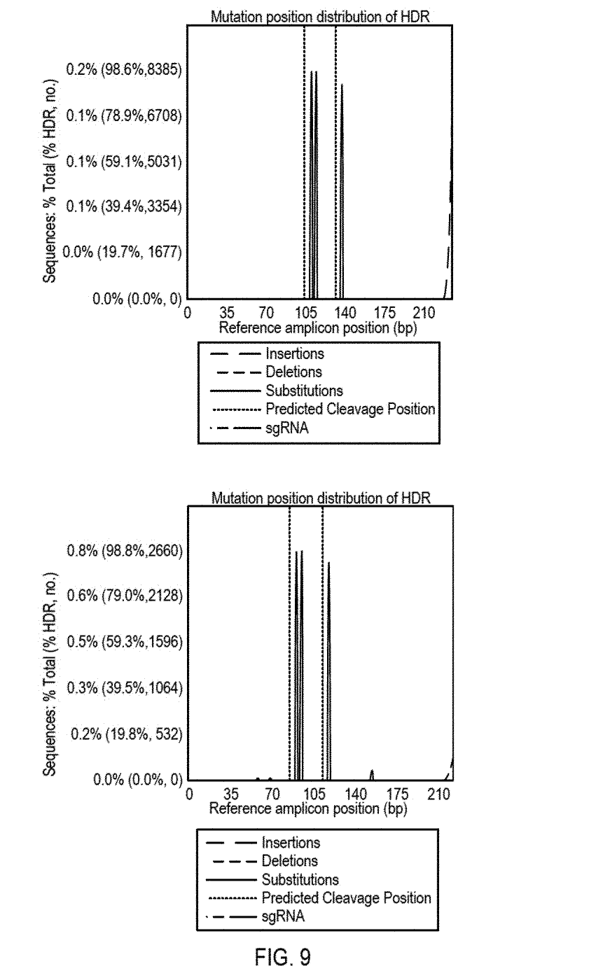

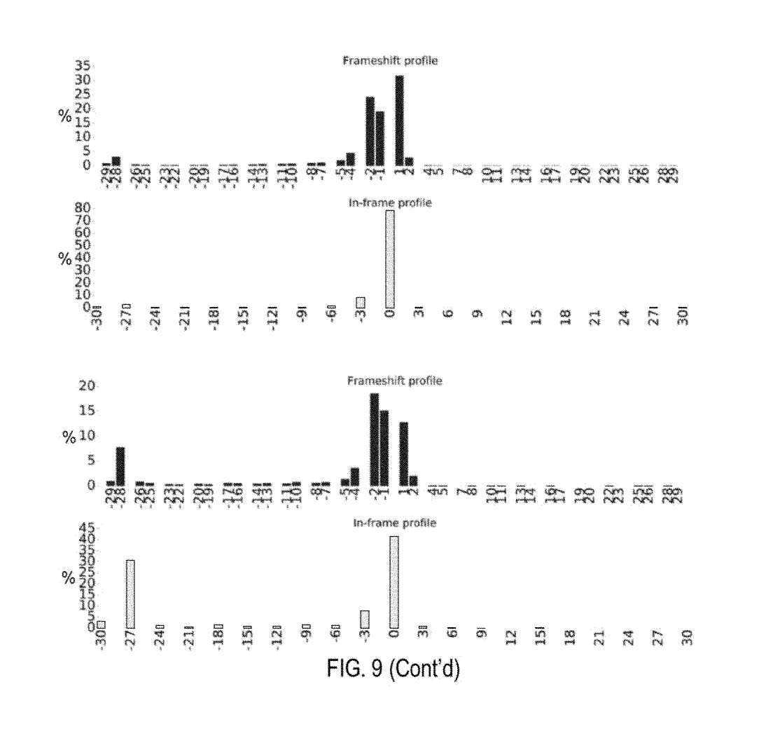

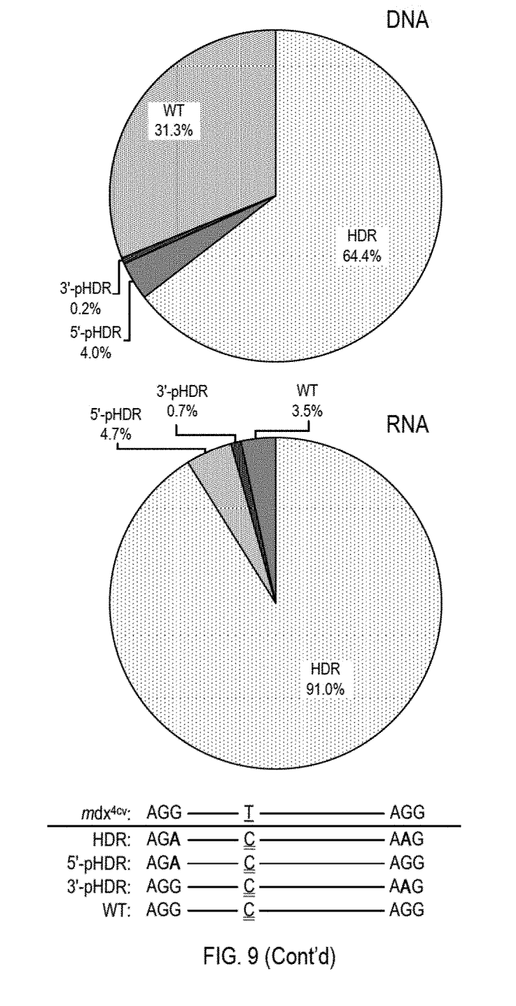

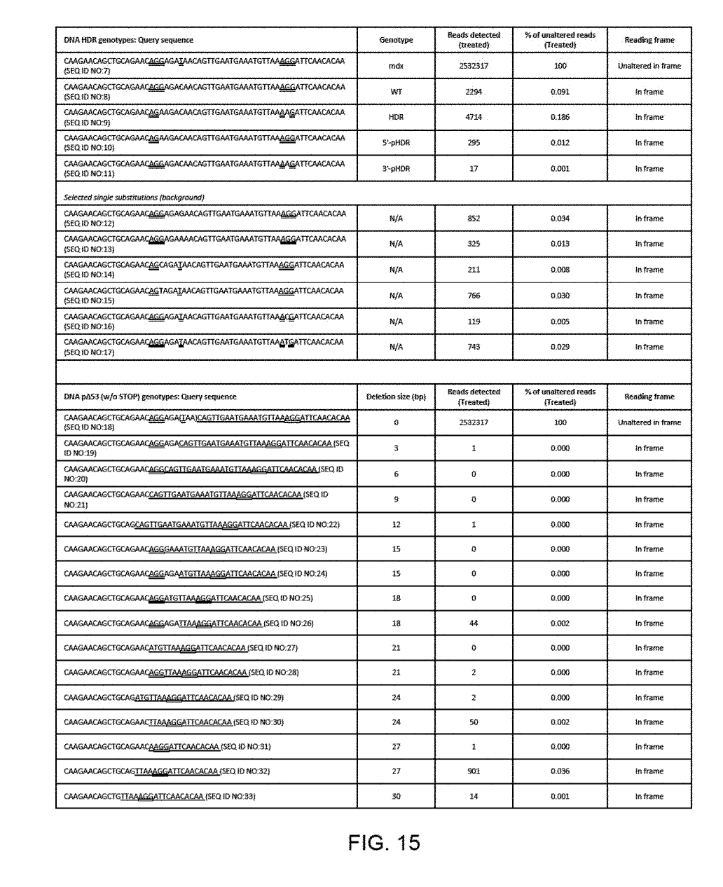

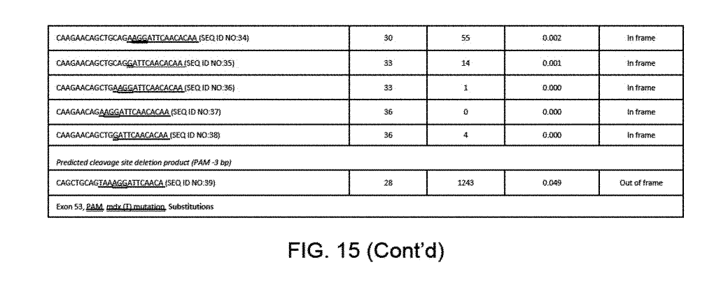

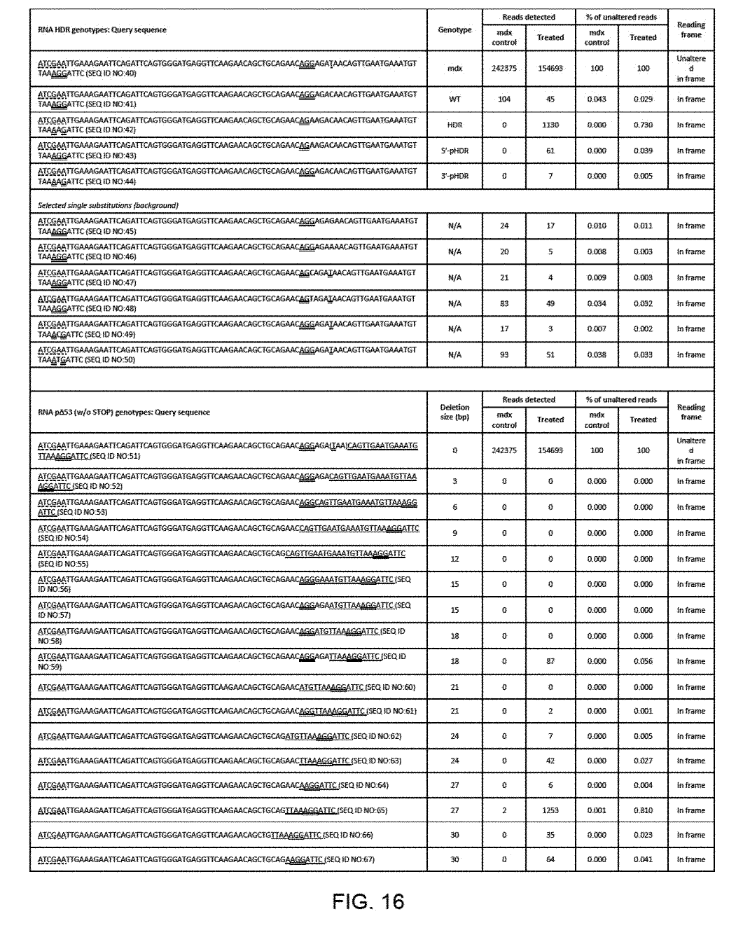

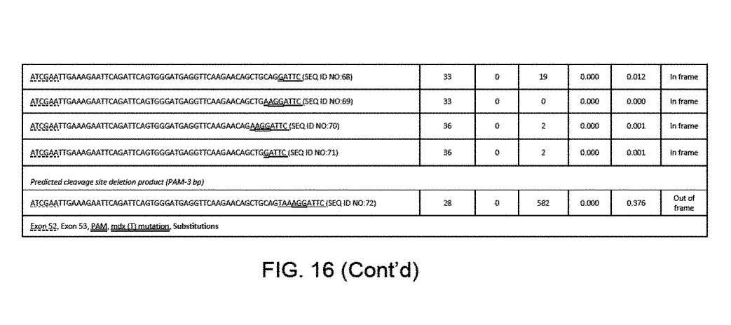

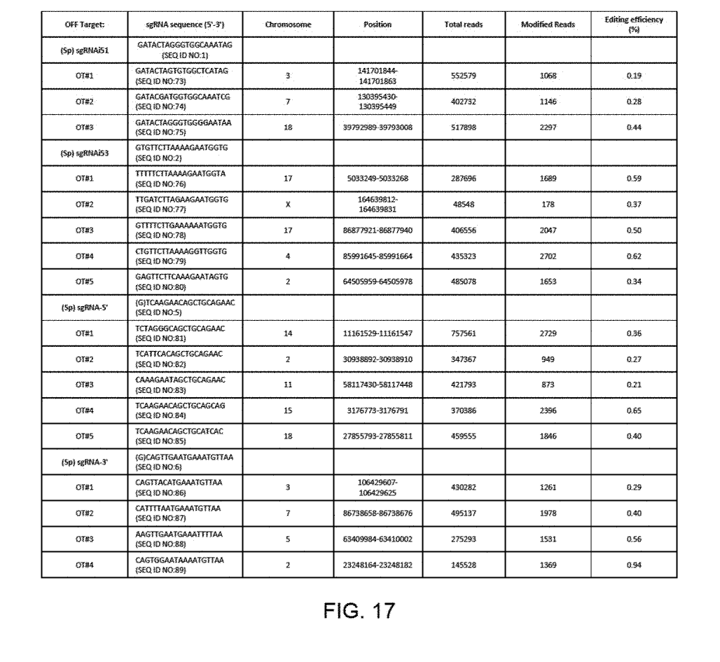

For strategy 2, the combined gene editing efficiency for both target sites within exon 53 was 2.3%, as determined by deep sequencing (see FIG. 2, panel D; FIG. 8, panels A-D; and Table 1). Encouragingly, successful HDR was detected in 0.18% of total genomes (see FIG. 2, panel D; FIGS. 9 and 15, and Table 1). While this efficiency was low (˜8% of the edited genomes resulted from HDR), the data show that myogenic cells within dystrophic muscles are at least modestly amenable to HDR-mediated dystrophin correction following CRISPR/Cas9 targeting. Analysis of dystrophin transcripts isolated from four treated samples revealed a unique shorter RT-PCR product that, following sequencing of individual cloned RT-PCR products, was shown to correspond to a complete deletion of exon 53 (see FIG. 8, panels A-D). This unanticipated exclusion of exon 53 from the mRNA likely resulted from larger indel mutations disrupting splicing enhancer signals located within this exon (see Ito, T., et al. Kobe J. Med. Sci. 47, 193-202 (2001)). Successful editing within the main exon 53 RT-PCR product was detected via both T7 endonuclease 1 digestion and Sanger sequencing of individual clones (see FIG. 8, panels A-D). Deep sequencing of RT-PCR amplicons spanning exons 52 and 53 revealed an overall editing efficiency of 9.2% at the transcript level with 0.8% of total transcripts corresponding to successful HDR events (see FIG. 2, panel D; FIGS. 8, 9, and 18; and Table 1), thus indicating successful Dmd gene editing and HDR within exon 53. Analysis of the sequence reads revealed several types of editing events. For example, 44% (genomic DNA) and 36% (mRNA) of the edited sequences carried insertions, deletions, or substitutions that did not shift the reading frame (see FIG. 2, panel E). However, only 3% (genomic DNA) and 16% (mRNA) of all edited sequences were in-frame deletions that also removed the mdx^{4cv} stop codon. Since ˜8% of all edited genomes and ˜9% of all edited transcripts resulted from HDR (see FIG. 2, panels D and E), a total of ˜11% (genomic) and ˜25% (transcript) of the strategy 2 editing events were able to express dystrophin (see FIG. 2, panel E; FIGS. 9, 15, and 16; and Table 1). Overall, on-target editing frequency was significantly higher than for predicted off-target sites sharing the most sequence similarity to the sgRNAs used in strategies 1 and 2 (see FIG. 17).

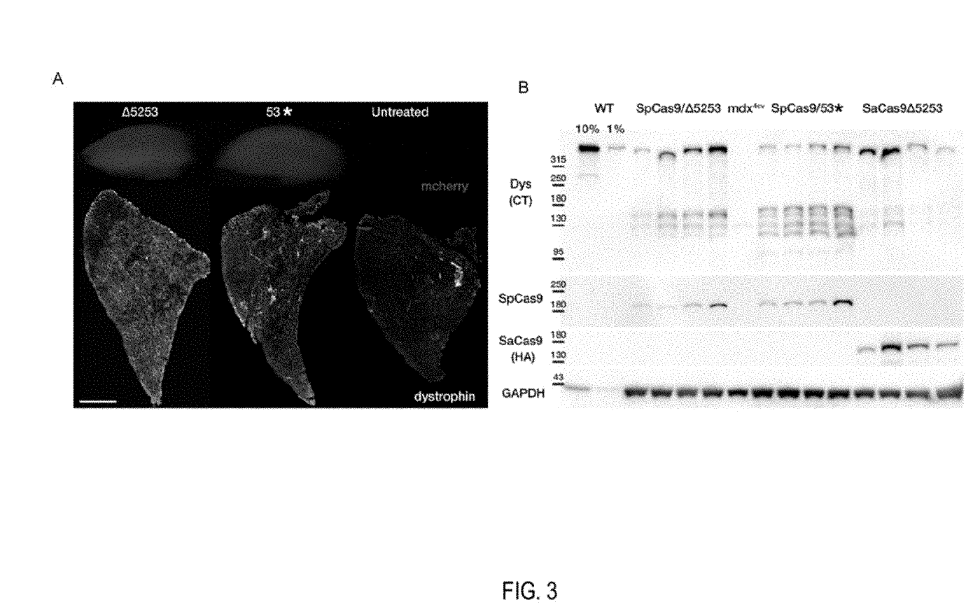

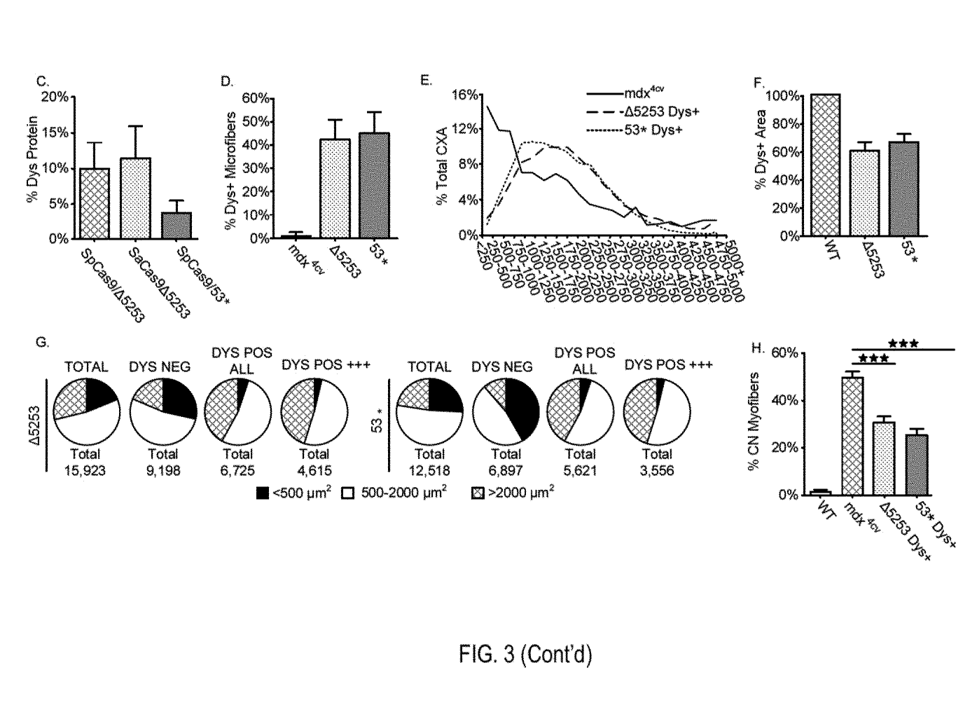

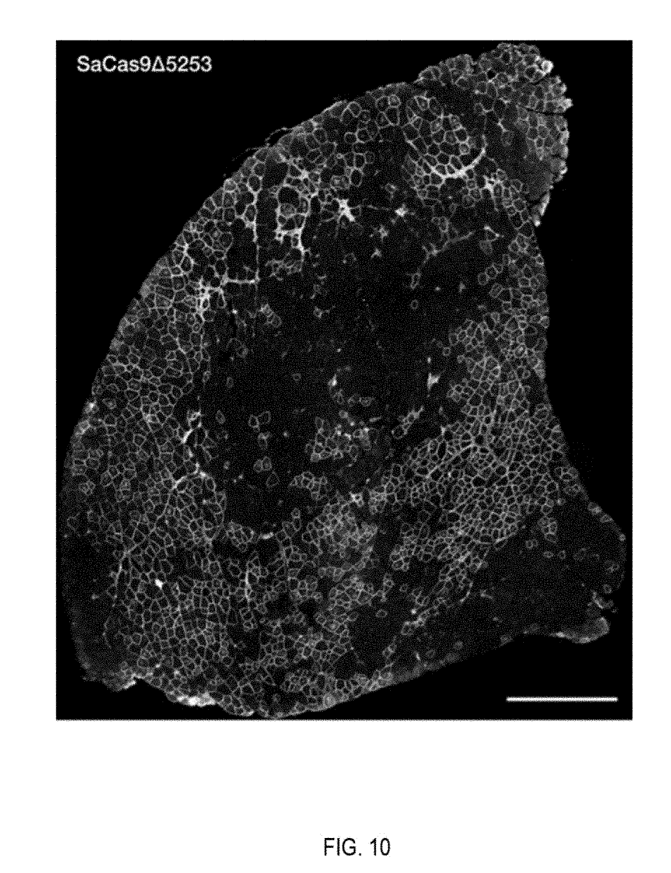

Establishment of a functional ORF led to significant induction of dystrophin expression in treated TAs as detected by immunostaining of muscle cryosections (see FIG. 3, panel A and FIG. 10) and by western blotting of whole muscle lysates (see FIG. 3, panel B). CRISPR/Cas9-mediated gene correction resulted in full- to near-full-length dystrophin protein expression levels of 0.8-18.6% (dual vector, n=4) or 1.5-22.9% (single vector, n=4) for strategy 1 and 1.8-8.4% (53*, dual vector, n=4) for strategy 2, as compared with wild-type (WT) dystrophin levels (see FIG. 3, panel C). In addition to the detection of full- to near-full-length dystrophin, western analysis also revealed a range of shorter dystrophin isoforms (110-160 kDa) of unclear therapeutic impact that were more frequent in strategy 2-treated muscles, possibly due to aberrant splicing.

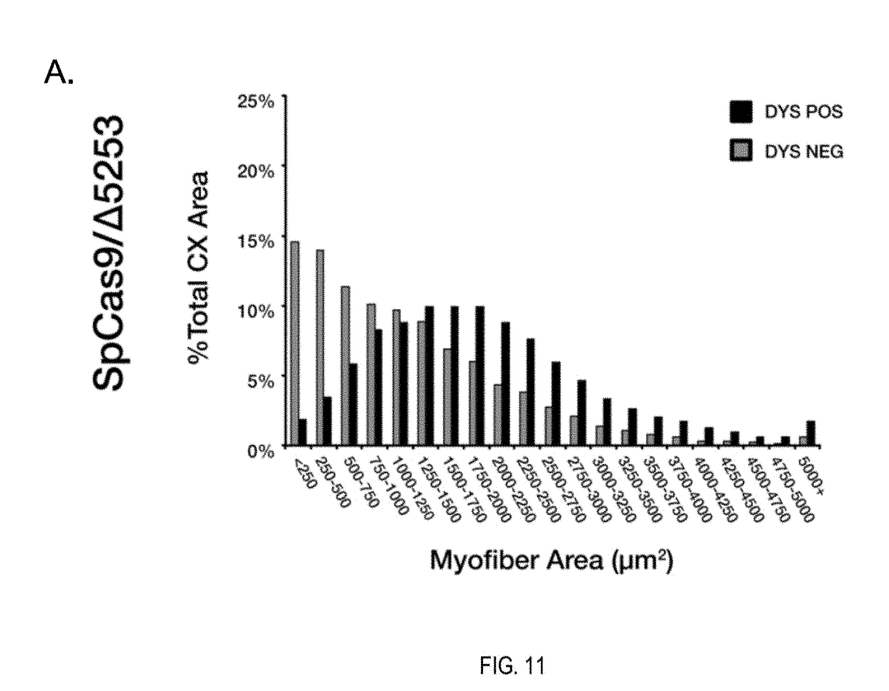

Immunostaining of muscle cross-sections revealed that an average of 41 (Δ5253) and 45% (53*) of myofibers expressed dystrophin (see FIG. 3, panel D). Of note, dystrophin-positive myofibers in treated TAs were significantly larger than myofibers of untreated mdx^{4cv} controls and than dystrophin-negative fibers within treated muscles (see FIG. 3, panels E and G and FIG. 11, panels A and B), constituting an average of 54% (Δ5253) and 61% (53*) of the myogenic cross-sectional area with a maximum observed positive area of 68% (Δ5253) and 71% (53*). Dystrophin-positive myofibers within treated muscles also displayed a significant reduction in central nucleation (see FIG. 3, panel H).

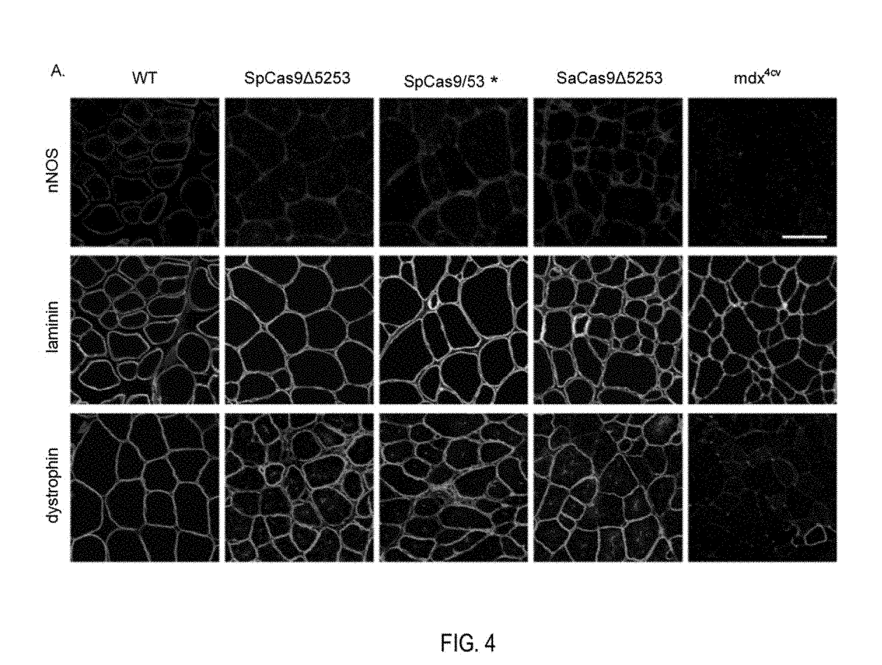

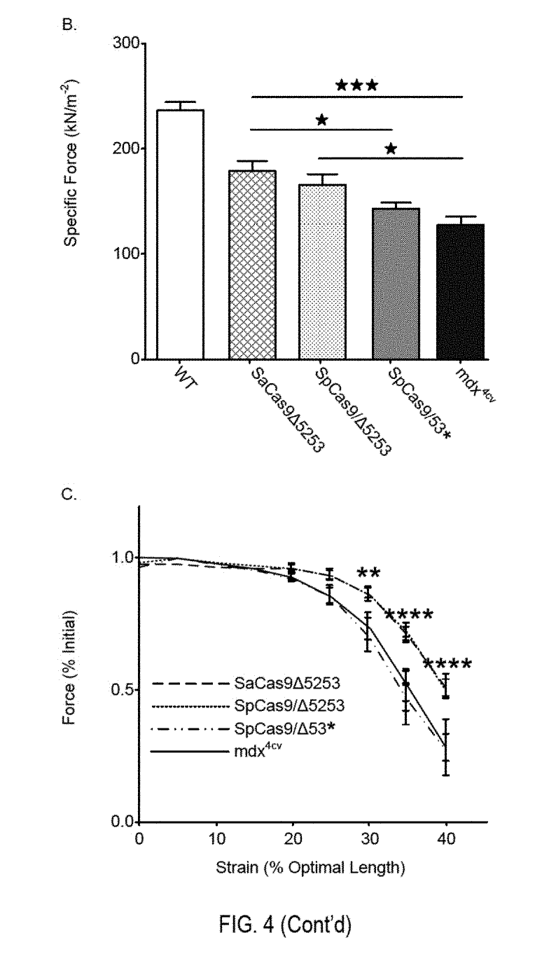

Induction of dystrophin expression also allowed for sarcolemmal localization of neuronal nitric oxide synthase (nNOS), an important component of the dystrophin-glycoprotein complex that modulates muscle performance (see FIG. 4, panel A) (see Lai, Y., et al. J. Clin. Invest. 119, 624-635 (2009)). To assess whether CRISPR/Cas9-mediated induction of dystrophin expression would translate into functional improvements in situ measurements of muscle force generation were performed at 18 weeks post-transduction of 2-week-old male mdx^{4cv} mice. Encouragingly, the observed dystrophin levels in muscles treated using strategy 1 were maintained at this later time point, resulting in significant increases in specific force generating capacity and protection from contraction-induced injury (see FIG. 4, panels B and C). Conversely, muscles treated according to strategy 2 only displayed a slight but non-significant increase in specific force development, likely due to the lower levels of dystrophin production.

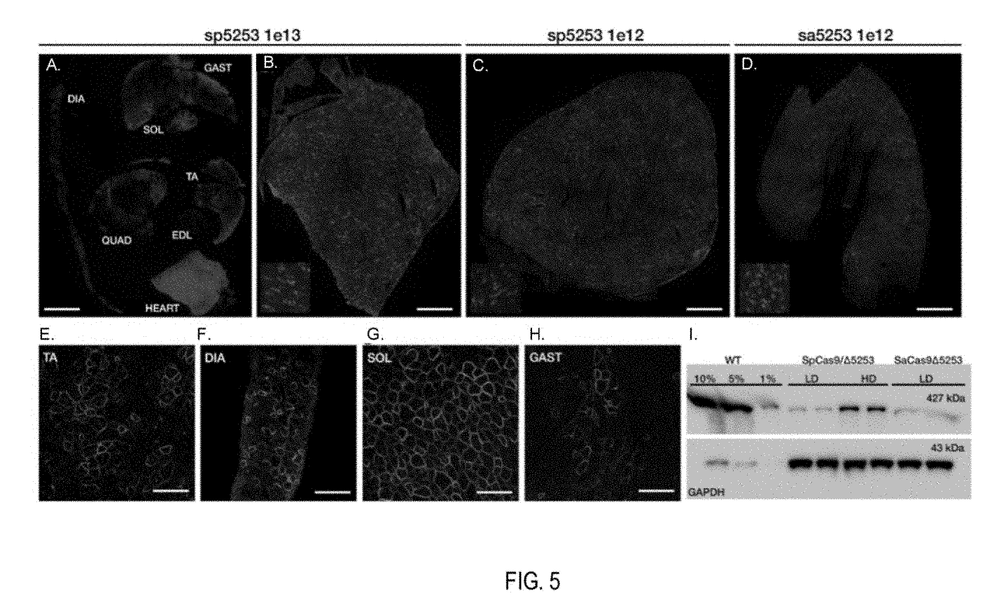

On the basis of the higher dystrophin-correction efficiency observed for strategy 1, this approach was tested following systemic delivery of the AAV nuclease and targeting vectors using a range of doses between 1-10×10¹² v.g. per mouse. Both single- and dual-vector approaches yielded widespread dystrophin expression in the heart, with up to 34% of cardiac myofibers expressing dystrophin at 4 weeks post-transduction (see FIG. 5). While both high- and low-vector doses were able to generate dystrophin expression in the heart (see FIG. 5, panel B-D), only the high dose was able to generate widespread, albeit variable, dystrophin expression in all muscle tissues analyzed (ranging from <10% dystrophin-positive fibers in the quadriceps and EDL muscles to >50% in soleus muscles; see FIG. 5, panel E-H). Furthermore, higher cardiac dystrophin expression levels were also obtained with increasing vector dose (see FIG. 5, panel I).

The results provided herein demonstrate that muscle-specific CRISPR/Cas9-mediated gene editing is effective in inducing dystrophin expression in dystrophic mdx^{4cv} mouse muscles. Localization of dystrophin-associated proteins, such as nNOS, to the sarcolemma and increased muscle force generation was also observed. Restriction of Cas9 expression to myogenic cells offers several advantages over ubiquitous expression by preventing expression of the bacterial Cas9 nuclease in non-muscle (including immune effector) cells and eliminating the impact of possible off-target events affecting genes expressed in mitotically active non-muscle cells, such as hepatocytes. Although HDR is believed to occur infrequently in post-mitotic tissues, at least a fraction of myogenic cells in dystrophic muscles displayed successful HDR-mediated gene correction following CRISPR/Cas9 delivery, as demonstrated by the presence of HDR-derived transcripts. Whether targeting of post-mitotic myonuclei or proliferating myogenic progenitors is responsible for these HDR events is currently unclear. However, MCK regulatory regions are not transcriptionally active in satellite cells or proliferating myoblasts (see Hu, C., et al. Mol. Ther. 22, 1792-1802 (2014); Chamberlain, J. S., et al. Mol. Cell. Biol. 5, 484-492 (1985); Jaynes, J. B., et al. Mol. Cell. Biol. 6, 2855-2864 (1986); and Hauser, M. A., et al. Mol. Ther. 2, 16-25 (2000)). In this regard, it was previously shown that homologous recombination between separate AAV vector genomes occurs at a moderate frequency in post-mitotic mouse myofibers (see Odom, G. L., et al. Mol. Ther. 19, 36-45 (2011)). Further improvements to HDR-based gene editing strategies could possibly be achieved by inhibiting genes involved in NHEJ, which may increase the efficiency of precise gene editing if the HDR events were occurring in mitotically active myogenic precursors (see Maruyama, T., et al. Nat. Biotechnol. 33, 538-542 (2015)), and/or via the use of alternative CRISPR associated nucleases (such as Cpf1 or Cas9-nickase) (see Zetsche, B., et al. Cell 163, 759-771 (2015) and Ran, F. A., et al. Cell 154, 1380-1389 (2013)).

For excision of exons 52-53, both single- and dual-vector approaches were able to induce dystrophin expression with similar efficiencies, despite an apparent higher frequency of editing with the dual vectors. It is possible that the difference in overall gene editing efficiency stems from a difference in the propensity for indel formation between SpCas9 and SaCas9 following DNA cleavage at the chosen target sites. For instances when DNA cleavage did not result in deletion of the intervening 45 kb segment, SpCas9 may have generated indels at the cut sites at higher frequencies than SaCas9, resulting in a perceived higher editing efficiency. Actual deletion of the intervening sequence may in fact have been comparable, which the downstream (mRNA and protein) data reflect. Nevertheless, a dual-vector approach may currently offer more flexibility in terms of allowing for variations in the ratio between administered nuclease versus targeting components, which may prove important for efficiency. If efficient transduction of myogenic stem cells (satellite cells) can be achieved in vivo, dystrophin correction could be permanent by ensuring continued generation of dystrophin expressing myofibers during normal muscle turnover. While previous results indicated that satellite cell transduction using AAV6, 8, or 9 is very low compared with myofibers (see Arnett, A. L. H., et al., Mol. Ther. Methods Clin. Dev. 1, 14038 (2014)), one other group found that AAV9 was able to target these stem cells with modest efficiency (see Tabebordbar, M., et al. Science 351, 407-411 (2016)). The reasons for these differing results are unclear, but significantly greater targeting efficiencies will likely be needed to support long-term regeneration from corrected myogenic stem cells. While the CK8 regulatory cassette in conjunction with CRISPR/Cas9 gene editing is clearly useful for correcting dystrophin mutations in myofibers, CK8 activity in satellite cells or proliferating myoblasts has not been observed (see Himeda, C. L., et al. Methods Mol. Biol. 709, 3-19 (2011) and Arnett, A. L. H., et al. Mol. Ther. Methods Clin. Dev. 1, 14038 (2014)).

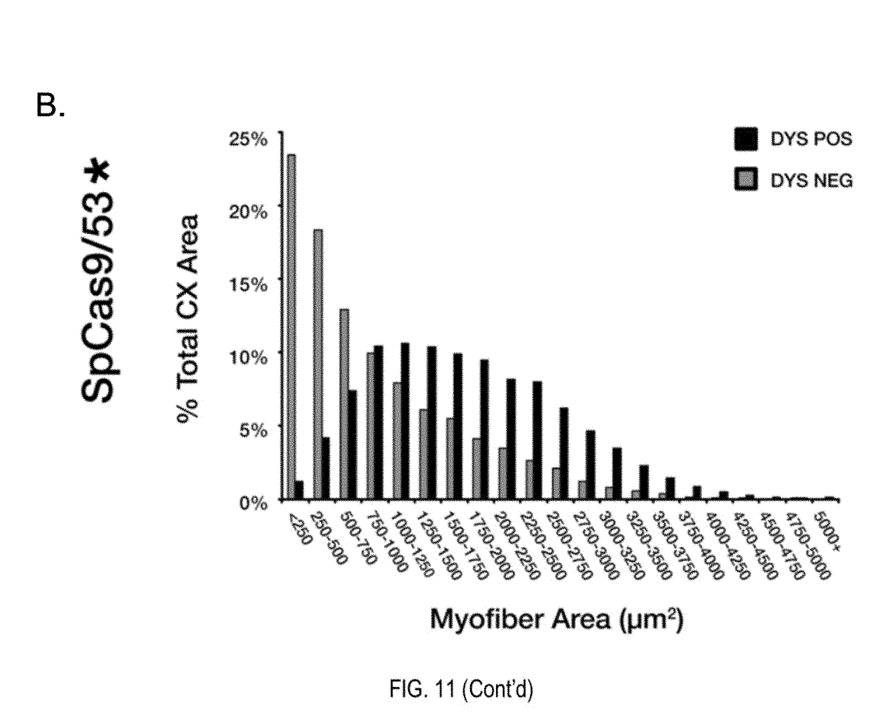

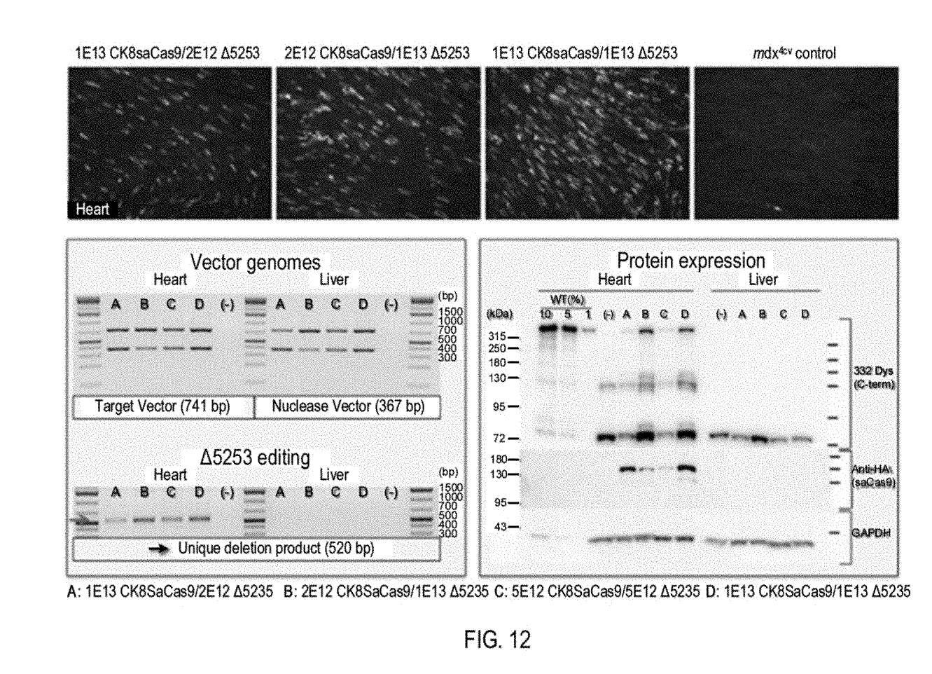

Immunofluorescent, DNA, and protein analyses at 12 weeks post systemic delivery of varying doses (A-D) of dual rAAV6 vectors consisting of a nuclease vector expressing SaCas9 under control of the muscle-specific CK8e promoter and a targeting vector (Δ5253) are depicted in FIG. 12. The targeting vector was designed to guide Cas9 to cut genomic DNA within the introns flanking exons 52 and 53, thereby removing exons 52-53 along with the premature stop codon responsible for the DMD phenotype and restoring an open reading frame encoding a functional dystrophin protein. Immunofluorescent analysis of cardiac cross-sections, as depicted in the top panels, show a dose dependent increase in dystrophin expressing cardiac myofibers. At the bottom left, DNA analysis by semi-quantitative PCR for the presence of AAV vector genomes in heart and liver show presence of vector genomes in both tissues (top panel). Semi-quantitative PCR reveals DNA targeting only in the heart based on the dose-dependent presence of a unique PCR product only generated upon removal of exons 52 and 53, ((-)=untreated control) (bottom panel). At the bottom right, western blotting analysis for protein expression demonstrates dose dependent dystrophin (top) and Cas9 (middle) expression exclusively in the heart. GAPDH loading control (bottom).

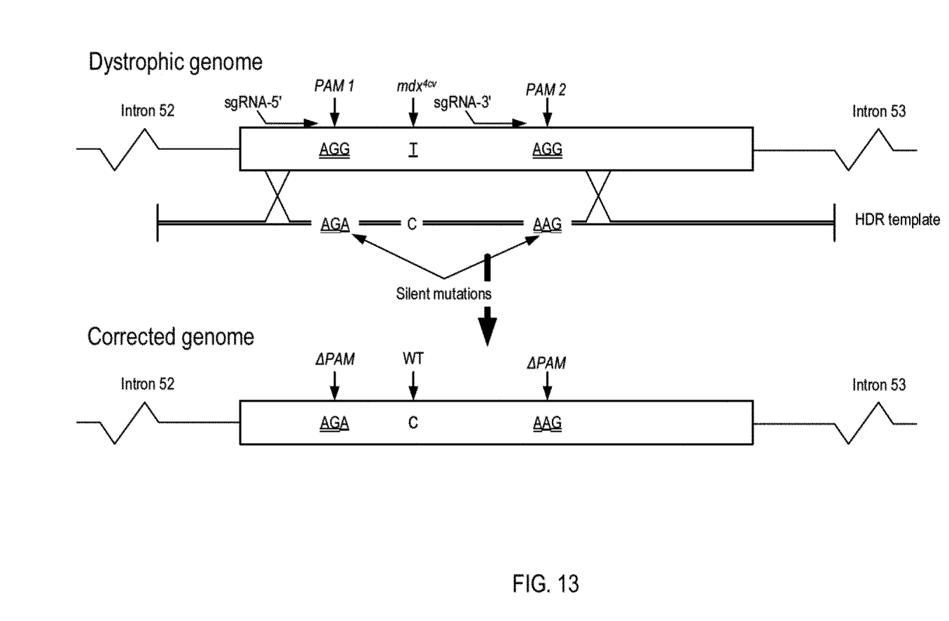

An overview of the HDR strategy provided herein is depicted in FIG. 13. Depicted at the top is exon 53 of the dystrophic host genome with the selected Cas9 target sites (sgRNA-5′ and -3′) along with their corresponding PAM sites (AGG) flanking the DMD-causing C to T substitution. Also depicted is the donor DNA template containing the normal C instead of T along with silently mutated PAM (encoding the same amino acid) which reduces the ability of Cas9 to cleave the donor template. As illustrated at the bottom, following Cas9 mediated double-stranded DNA cleavage of the dystrophic host genome, HDR-mediated repair replaces the host (dystrophic) genomic region with the modified “normal” genome encoding full-length functional dystrophin.

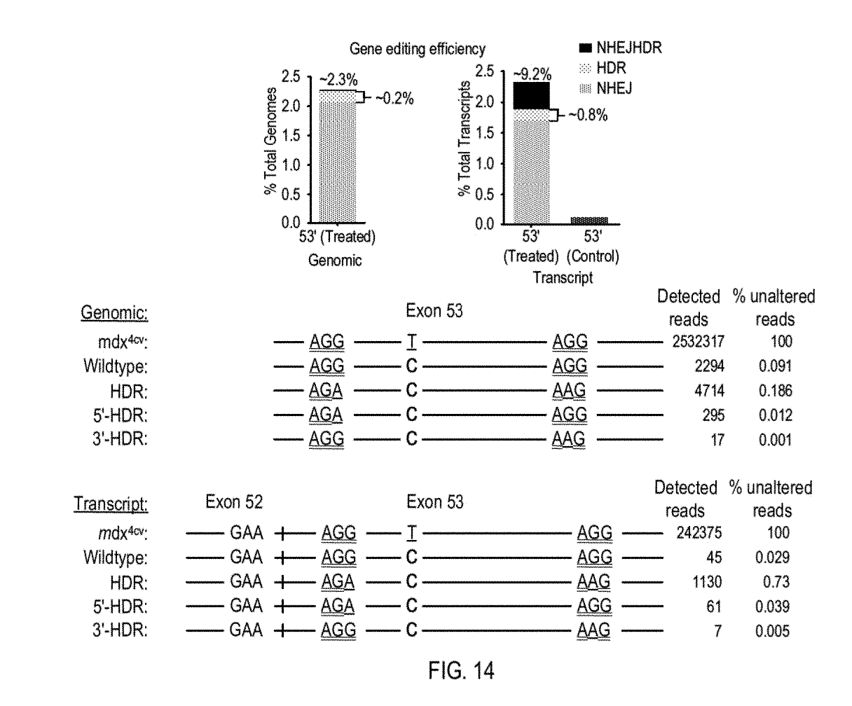

Analysis of gene editing efficiency and successful HDR at 4 weeks post-treatment is depicted in FIG. 14. At the top, next-generation sequencing of PCR amplicons generated from genomic DNA (left) and cDNA derived from mRNA (right) is shown. Genomic DNA showed an overall gene editing efficiency of ˜2.3% including insertion/deletion events repaired via NHEJ, HDR, and a mix of both. Approximately 0.2% of the total genomes corresponded to successful HDR. At the transcript level the overall editing efficiency rose to ˜9.2% with ˜0.8% corresponding the successful HDR (note: transcripts isolated from untreated control mice showed only background levels of editing). At the bottom, it is shown that manual analysis of detected genotypes at both genomic and transcript levels provides additional information about what portion of the donor DNA template was successfully integrated.

A first aspect of the disclosure relates to pharmaceutical or biopharmaceutical compositions. The pharmaceutical composition may include a muscle-specific nuclease cassette, one or more gRNA cassettes (e.g., a first gRNA cassette), and/or a mutation-corrected homology template (e.g., for HDR) and a delivery system for delivery of the muscle-specific nuclease cassette, the gRNA cassette(s), and/or the mutation-corrected homology template.

In some embodiments, the muscle-specific nuclease cassette may include a muscle-specific transcriptional regulatory cassette and a nuclease coding sequence. The nuclease coding sequence may encode a CRISPR-associated nuclease. For example, the nuclease coding sequence may encode a protein selected from SaCas9, SpCas9, Cpf1, or another suitable CRISPR-associated nuclease.

In some embodiments, the muscle-specific transcriptional regulatory cassette may be derived from an M-creatine kinase enhancer and/or a M-creatine kinase promoter sequence. For example, the muscle-specific transcriptional regulatory cassette may be derived from a M-creatine kinase enhancer plus a M-creatine kinase promoter. Furthermore, the muscle-specific transcriptional regulatory cassette may include one or more enhancers derived from conserved regions of muscle creatine kinase and/or a CK8 transcriptional regulatory cassette (SEQ ID NO:159).

The muscle-specific transcriptional regulatory cassette may be a muscle-specific CK8 transcriptional regulatory cassette (CK8). CK8 is a non-naturally occurring nucleotide sequence including multiple muscle and non-muscle gene control elements arranged in a miniaturized array. CK8 may provide high or very high transcriptional expression of a predetermined RNA and/or protein in skeletal and cardiac muscle cells.

In certain embodiments, the muscle-specific transcriptional regulatory cassette may be a CK8 transcriptional regulatory cassette. The CK8 transcriptional regulatory cassette may have at least 70% sequence identity, at least 80% sequence identity, at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the nucleic acid sequence of SEQ ID NO:159.

In various embodiments, the muscle-specific transcriptional regulatory cassette may express the nuclease coding sequence such that a level of expression of the nuclease coding sequence is at least 50-fold higher, at least 75-fold higher, at least 100-fold higher, or at least 150-fold higher in muscle cells than the level of expression of the nuclease coding sequence in non-muscle cells.

The pharmaceutical composition may further include a second gRNA cassette, wherein the first gRNA cassette includes a first gRNA coding sequence and the second gRNA cassette includes a second gRNA coding sequence. In some other embodiments, the pharmaceutical composition may further include three or more gRNA cassettes. For example, the pharmaceutical composition may further include: a third gRNA cassette, wherein the third gRNA cassette includes a third gRNA coding sequence; a fourth gRNA cassette, wherein the fourth gRNA cassette includes a fourth gRNA coding sequence; and so on.

In certain embodiments, the pharmaceutical composition may further include a mutation-corrected DNA template, wherein the mutation-corrected DNA template is configured for HDR. The muscle-specific transcriptional regulatory cassette and/or the gRNA cassettes described above may also include such a mutation-corrected DNA template (or the mutation-corrected DNA template may be delivered separately from the muscle-specific transcriptional regulatory cassette and/or the gRNA cassettes), wherein the mutation-corrected DNA template may be configured for HDR. The mutation-corrected DNA template may be configured to repair a mutated target nucleic acid sequence. In some embodiments, the mutated target nucleic acid sequence may be in a gene associated with a neuromuscular disorder. For example, the mutated target nucleic acid sequence may be in a gene encoding dystrophin.

In some embodiments, the delivery system can include a recombinant adeno-associated virus (rAAV) vector. For example, the rAAV vector may be an rAAV6 vector, an rAAV8, an rAAV9 vector, or another suitable rAAV vector. In various embodiments, the rAAV vector may be an rAAV6 vector. The delivery system may include a single rAAV vector to deliver the muscle-specific nuclease cassette and the one or more gRNA cassettes. Alternatively, the delivery system may include a first rAAV vector to deliver the muscle-specific nuclease cassette and a second rAAV vector to deliver the one or more gRNA cassettes. Furthermore, the delivery system may include a third rAAV vector to deliver an additional gRNA cassette, a fourth rAAV vector to deliver an additional gRNA cassette, and so on. Any of these rAAV vectors may include a mutation-corrected DNA template configured for HDR.

The pharmaceutical composition may reduce a pathological effect or symptom of a neuromuscular disorder in a subject. In various embodiments, the pharmaceutical composition may increase a specific-force generating capacity of at least one skeletal muscle in a subject to within at least 25%, at least 30%, at least 40%, or at least 50% of a normal specific-force generating capacity in a skeletal muscle. In some embodiments, the pharmaceutical composition may restore a baseline end-diastolic volume defect in a subject to within at least 25%, at least 30%, at least 40%, or at least 50% of a normal end-diastolic volume.

The pathological effect or symptom of the neuromuscular disorder may be selected from at least one of muscle pain, muscle weakness, muscle fatigue, muscle atrophy, fibrosis, adipose cell accumulation, inflammation, increase or decrease in average myofiber diameter in skeletal muscle, centrally-nucleated myofiber number, cardiomyopathy, reduced 6-minute walk test time, loss of ambulation, and cardiac pump failure.

The neuromuscular disorder may be a muscular dystrophy selected from at least one of myotonic muscular dystrophy (DM1 and/or DM2), Duchenne muscular dystrophy, Becker muscular dystrophy, any of the various types of limb-girdle muscular dystrophy, facioscapulohumeral muscular dystrophy, any of the various types of congenital muscular dystrophy, oculopharyngeal muscular dystrophy, distal muscular dystrophy, desmin-related myopathies, fukyama muscular dystrophy, FKRP-deficiencies and Emery-Dreifuss muscular dystrophy. In some embodiments, the muscular dystrophy may be Duchenne muscular dystrophy.

Another aspect of the disclosure relates to methods for treating a subject having a neuromuscular disorder. In certain embodiments, the method may include administering to the subject a therapeutically effective amount of a pharmaceutical composition. The pharmaceutical composition may include a muscle-specific nuclease cassette and one or more gRNA cassettes and/or a mutation corrected template for HDR. The pharmaceutical composition may further include a delivery system for delivery of the muscle-specific nuclease cassette, the one or more gRNA cassettes, and/or the mutation-corrected DNA template configured for HDR.

In various embodiments, the therapeutically effective amount of the pharmaceutical composition may be between about 10¹¹ and about 10¹⁶ vector genomes (vg)/kilogram (kg) subject weight, between about 10¹² and about 10¹⁵ vg/kg subject weight, between about 10¹³ and about 10¹⁴ vg/kg subject weight, or another suitable amount. In some embodiments, the pharmaceutical composition may be administered intravascularly, intraperitoneally, subcutaneously, or orally. In certain embodiments, the pharmaceutical composition may include no, up to 5%, up to 10%, up to 20%, up to 30%, up to 40%, up to 50%, up to 60%, up to 70%, up to 80%, or up to 90% empty capsids (see U.S. Pat. No. 7,655,467 and European Patent No. 1689230).

Another aspect of the disclosure relates to methods of modifying the sequence of a target nucleic acid sequence in a muscle cell or a myogenic progenitor cell. In certain embodiments, the method may include contacting or transducing the muscle cell or the myogenic progenitor cell with a delivery system and/or the contents of the delivery system. The delivery system may include a muscle-specific nuclease cassette, one or more gRNA cassettes, and/or a mutation-corrected DNA template comprising a modification to be made in the target nucleic acid sequence (i.e., a homology template for HDR).

In certain embodiments, the method may include inducing expression of a gene associated with a neuromuscular disorder in the muscle cell. For example, the method may include inducing expression of dystrophin in the muscle cell.

### EXAMPLES

The following examples are illustrative of disclosed methods and compositions. In light of this disclosure, those of skill in the art will recognize that variations of these examples and other examples of the disclosed methods and compositions would be possible without undue experimentation.

### Example 1

### Cloning and Vector Production

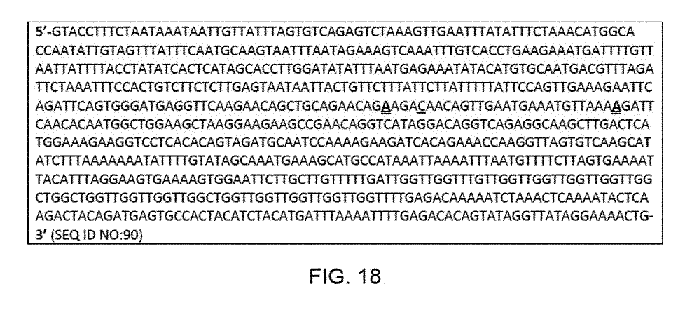

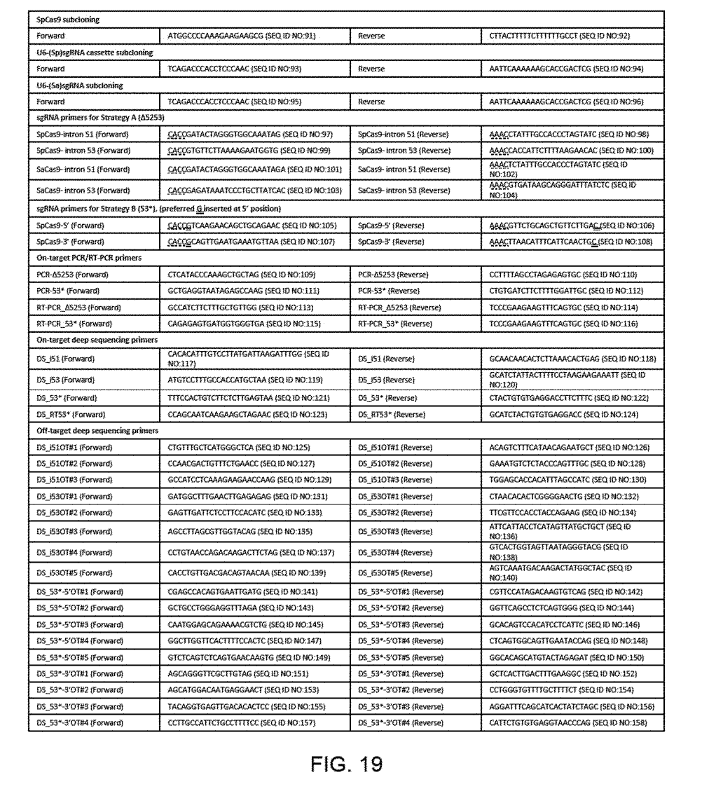

Plasmids containing regulatory cassettes for expression of Cas9 or gRNAs flanked by AAV serotype 2 inverted terminal repeats (ITRs) were generated using standard cloning techniques. The spCas9 nuclease expression cassette was generated by PCR cloning of NLS-SpCas9-NLS from LentiCRISPRv1 (see Shalem, O., et al. Science 343, 84-87 (2014)), and insertion into pAAV (STRATAGENE™) containing the ubiquitous elongation factor-1 alpha short promoter (EFS) (id.) (for in vitro studies in fibroblasts) or the muscle-specific creatine kinase 8 (CK8) regulatory cassette (RC) (see Himeda, C. L., et al. Methods Mol. Biol. 709, 3-19 (2011) and Hu, C., et al. Mol. Ther. 22, 1792-1802 (2014)) (for in vivo studies). (Sp)sgRNA target sequences were selected using the online software ZIFIT TARGETER™ (http://zifit_partners_org/ZiFiT/) and inserted into pLentiCRISPRv1 following BsmB1 restriction enzyme digestion. Two targeting constructs to work in conjunction with SpCas9 were generated by PCR cloning of the U6-(Sp)sgRNA expression cassette from pLentiCRISPRv1 followed by insertion into pAAV plasmids on either side of a CMV-mCherry expression cassette and a HDR template spanning positions X84575274 to X84576081 of the murine DMD gene cloned from C57BL/6 genomic DNA. The corresponding protospacer adjacent motif (PAM) sites at positions X84575612 (G-A) and X84575639 (G-A) within the HDR template were mutated using PCR-mediated mutagenesis while preserving the encoded amino acids (silent mutations) to eliminate or reduce targeting of the DNA repair template by Cas9. The modified HDR sequence, gRNA sequences as well as primer sequences for cloning and PCR amplification of genomic DNA and complementary DNA (cDNA) are provided in FIGS. 18 and 19. The SaCas9 single vector expression cassette was generated by replacing the CMV immediate early enhancer and promoter and the bovine growth hormone poly-adenylation (pA) signal in plasmid #61591 (ADDGENE™) (see Ran, F. A., et al. Nature 520, 186-191 (2015)) with the CK8 RC and a rabbit beta-globin pA signal, followed by PCR cloning and insertion of a second U6-(Sa)sgRNA expression cassette sequential to the first. (Sa)sgRNA target sequences were manually selected to target the same locations as the (Sp)sgRNAs and inserted into the U6-(Sa)sgRNA expression cassette via Bsa1 restriction enzyme digestion before inserting the second U6-(Sa)sgRNA cassette into the final construct. Nuclease and targeting pAAV plasmids were co-transfected with the pDG6 packaging plasmid into subcultured HEK293 cells (AMERICAN TYPE CULTURE COLLECTION®) using calcium phosphate-mediated transfection to generate AAV6 vectors that were harvested, purified via heparin-affinity chromatography and concentrated using sucrose gradient centrifugation (see Blankinship, M. J., et al. Mol. Ther. 10, 671-678 (2004)). Resulting titers were determined by Southern analyses using probes specific to the poly-adenylation signal or CMV promoter for nuclease and targeting vectors, respectively.

### Example 2

### Electroporation and Culture of Primary Dermal Fibroblasts

Primary dermal fibroblasts were isolated from 3-week-old male mdx^{4cv} mice (see Takashima, A. Curr. Protoc. Cell Biol. 2.1, 2.1.1-2.1.12 (2001)). Electroporation of ˜600,000 cells per strategy were performed in INVITROGEN™ R-buffer containing 4 μg of both nuclease (EFS-SpCas9) and targeting (Δ5253/53*) plasmid expression constructs using a NEON® transfection system (INVITROGEN™) with three 10 ms pulses of 1,650 volts. Cells were subsequently seeded on 0.1% gelatin-coated culture vessels and maintained for 12 days in Dulbecco's modified Eagle medium supplemented with Penicillin-Streptomycin, Sodium pyruvate, L-Glutamine and 15% fetal bovine serum (THERMO FISHER SCIENTIFIC™) before harvest and DNA isolation (DNEASY° , QIAGEN™)

### Example 3

### Animals

All animal experiments were approved by the Institutional Animal Care and Use Committee of the University of Washington. Intramuscular delivery of 2.5-5×10¹⁰ v.g. of each vector (nuclease and targeting) was performed via longitudinal injection into tibialis anterior (TA) muscles of 2-12-week-old male C57BL/6-mdx^{4cv} (mdx^{4cv}) mice. For strategy 1, systemic delivery of 1×10¹² v.g. (low dose) to 1×10¹³ v.g. (high dose) was performed via retro-orbital injection into 11 week-old male mdx^{4cv} mice (n=3). Both dual- and single-vector approaches were evaluated at the low dose of 1×10¹² v.g. of each vector, while the dual-vector approach was also evaluated at a high dose of 1×10¹³ v.g. of the nuclease vector and 4×10¹² v.g. of the targeting vector. The mdx^{4cv} mouse model of DMD harbors a nonsense C to T mutation in exon 53 leading to a loss of dystrophin expression (see Im, W. B., et al. Hum. Mol. Genet. 5, 1149-1153 (1996)). These mice exhibit ˜10-fold lower frequencies of revertant dystrophin expressing muscle fibers than the original mdx^{scsn} mouse strain, which provides much greater assurance that dystrophin-corrected fibers resulted from gene targeting rather than spontaneous reversion.

### Example 4

### Tissue Harvest and Processing

Muscles were collected and analyzed at 4 weeks post-transduction and compared with age-matched male non-injected mdx^{4cv} and WT mice, except for mice undergoing physiological measurements which were analyzed at 18 weeks post-transduction. Medial portions of TA muscles were embedded in Optimal Cutting Temperature (O.C.T.) compound (VWR® International) and fresh frozen in liquid nitrogen cooled isopentane for immunofluorescence analysis. The remaining portions of TA muscles were snap frozen in liquid nitrogen and ground to a powder under liquid nitrogen in a mortar kept on dry ice for subsequent extraction of DNA, RNA, and protein.

### Example 5

### Immunohistochemical and Morphometric Analyses

TA cross-sections (10 μm) were co-stained with antibodies raised against alpha 2-laminin (SIGMA®, rat monoclonal, 1:200) and the C-terminal domain of dystrophin (from Dr. Stanley Froehner at the University of Washington, Department of Physiology and Biophysics, rabbit polyclonal, 1:500). Serial sections were stained with antibodies raised against neuronal nitric oxide synthase (INVITROGEN™, rabbit polyclonal, 1:200). Slides were mounted using PROLONG® Gold with DAPI (THERMO FISHER SCIENTIFIC™) and imaged via LEICA™ SPV confocal microscope at the University of Washington Biology Imaging Facility (http://depts_washington_edu/if/). Confocal micrographs covering the entirety of injected TA muscle sections were acquired and montaged using the FIJI™ toolset (IMAGEJ™) and PHOTOSHOP® (ADOBE™). Quantification of dystrophin-positive myofibers and dystrophin-positive muscle cross-sectional area was performed via semi-automated tracing and measurement of 1,250 to 3,500 individual myofibers per TA using ADOBE PHOTOSHOP® (n=5 TAs per treatment group). Automated quantification of central nucleation was performed using software developed in-house by Rainer Ng (CHAMP) running on the MATLAB™ platform.

### Example 6

### Nucleic Acid and Protein Analyses

DNA and RNA were isolated using TRIZOL® reagent (INVITROGEN™) according to the manufacturer's recommendations. Approximately 500 bp amplicons across the targeted regions of genomic DNA were generated by PCR using PHUSION® proof-reading polymerase (NEW ENGLAND BIOLABS®) and analyzed for targeting efficiency using T7 endonuclease 1 (NEW ENGLAND BIOLABS®), next generation sequencing (BGI™ International or in-house) or Sanger sequencing (SIMPLESEQ™, EUROFINS™ MWG Operon) of subclones of PCR amplicons (ZERO BLUNT™ TOPO™, INVITROGEN™). The T7 endonuclease assay was performed by denaturing and re-annealing the amplified PCR product followed by treatment with T7 endonuclease 1 to cleave indel-derived heteroduplex PCR products. Analysis of dystrophin-targeted transcripts by RT-PCR of the target regions was performed on cDNA made using SUPERSCRIPT® III first-strand synthesis superm ix (INVITROGEN™). Specific indel mutations or deletions in the dystrophin transcript were identified by Sanger sequencing of individual subclones of RT-PCR fragments. Muscle proteins were extracted in radioimmunoprecipitation analysis buffer (RIPA) supplemented with 5 mM EDTA and 3% protease inhibitor cocktail (SIGMA®, Cat #P8340), for 1 hour on ice with gentle agitation every 15 minutes. Total protein concentration was determined using PIERCE™ BCA assay kit (THERMO FISHER SCIENTIFIC™). Muscle lysates from WT (10 and 1 μg), untreated mdx^{4cv} (30 μg), and treated mdx^{4cv} (30 μg) mice were denatured at 99 degrees Celsius for 10 minutes, quenched on ice and separated via gel electrophoresis after loading onto BOLT™ 4-12% Bis-Tris polyacrylamide gels (INVITROGEN™). Protein transfer to 0.45 μm PVDF membranes was performed overnight at constant 34 volts at 4 degrees Celsius in Towbin buffer containing 20% methanol. Blots were blocked for 1 hour at room temperature in 5% non-fat dry milk before overnight incubation with antibodies raised against the C-terminal domain of dystrophin (Froehner Lab, Rabbit polyclonal, 1:10,000), anti-SpCas9 (MILLIPORE™, mouse monoclonal, 1:2,000), anti-HA (ROCHE™, Rat monoclonal-HRP conjugated, 1:2,000) for detection of HA-tagged saCas9 and GAPDH (SIGMA®, Rabbit polyclonal, 1:100,000). Horseradish-peroxidase conjugated secondary antibody staining (1:50,000) was performed for 1 hour at room temperature before signal development using CLARITY™ Western ECL substrate (BIORAD™) and visualization using a CHEMIDOC™ MP imaging system (BIORAD™) Gel- and blot-band densitometry measurements were performed on unsaturated images using IMAGEJ™ software (National Institutes of Health).

### Example 7

### Deep Sequencing

Approximately 200-250 bp PCR products were generated across target, and the top predicted off-target sites for each sgRNA using PLATINUM® Taq High-Fidelity polymerase (INVITROGEN™) or PHUSION® High-Fidelity Polymerase (NEW ENGLAND BIOLABS®). Potential off-target sites were identified using ZIFIT TARGETER™ software for SpCas9. CRISPR Rgen tools Cas-OFFinder software (http://www_rgenome_net/cas-offinder/) was used to identify potential off-target sites for SaCas9, using a mismatch number of ≦3, DNA bulge size ≦1 and RNA bulge size ≦1 For Strategy B, genomic deep sequencing was performed on a ˜230 bp nested PCR product generated from an initial ˜500 bp product amplified across exon spanning both target sites. To eliminate false detection of the HDR template DNA present in AAV vectors, the primer pair used to generate the 500 bp product was designed with one primer annealing outside of the region complimentary to the HDR template. The resulting PCR product was isolated following gel electrophoresis (GENEJET™ gel extraction kit, THERMO FISHER SCIENTIFIC™ ) before performing nested PCR followed by a second gel extraction. For each site analyzed, amplicons from 4-5 mice were pooled and subjected to standard ILLUMINA® library preparation (A-tailing, adaptor ligation, and amplification using NEBNEXT® library preparation kit (NEW ENGLAND BIOLABS®)), and QC'd using a BIOANALYZER™ before paired end (PE150) sequencing on an ILLUMINA® MISEQ™ system)(ILLUMINA® . Libraries were barcoded for multiplexed sequencing and subsequent reads were parsed and QC'd using custom scripts (TRIM GALORE™ software (http://www_bioinformatics_babraham_ac_uk/projects/trim_galore/), phred33 score≧30) and standard ILLUMINA® tools. On-target paired end (PE150) sequencing of DNA amplicons generated from muscles treated according to strategy 2 (53*) was performed by submitting the samples to BGI™ International (BGI™ AMERICAS). Uniquely mapping read pairs were used for downstream analysis using the CRISPRESSO™ software pipeline (see Pinello, L., et al. Nat. Biotechnol. 34, 695-697 (2016)). For CRISPRESSO™ analyses: 25 by at each end of the amplicon were excluded from quantification, the window size around each cleavage site used to quantify NHEJ events was set to 5 bp and sequence homology for an HDR occurrence was set to 98%. Following CRISPRESSO™ analysis, manual analysis and quantification was performed by searching for defined sequences in the quality-filtered and adapter-trimmed deep sequencing FASTQ files to provide further information on specific genotypes generated by strategy 2. For DNA reads, search sequences were chosen to span the region containing both target sites and the site of the C-T mutation. For RNA reads, search sequences were defined to span a region starting from within exon 52 (>45 kb away from the target region) extending past the prototypical cut site at the 3′ end of the target region.

### Example 8

### Statistical Analyses

Data values are represented as mean±s.e.m. and were analyzed in EXCEL™ (MICROSOFT™) and PRISM6™ (GRAPHPAD™). Measurements were analyzed for statistical significance using one-way analysis of variance (ANOVA) multiple comparison tests with Turkey's post hoc tests unless otherwise stated. Statistical significance was set to P<0.05.

### Example 9

### Comparison of SpCas9 to SaCas9 and SpCas9-HF1 (ADDGENE™ plasmid #72247)

The gene editing efficiency of SpCas9, SaCas9, and the new “HF” Cas9 can be compared. Efficiency can be assessed by injecting 12-week-old male mdx^{4cv} mouse TA muscles IM, and analyzing mice 1 and 2 months later. Each Cas9 vector can be co-delivered with a vector expressing single guide RNA expression cassettes (sgRNAs) targeting: a) introns 51 and 53 (to delete exons 52-53, a 45 kb genomic interval); b) the region adjacent to the mdx^{4cv} mutation (for NHEJ); or c) two regions flanking the mdx^{4cv} mutation along with a homology template (to measure HDR). Efficiency can be measured by deep sequencing of: a) PCR products spanning the targeted regions; b) RT-PCR products spanning exons 51-55; and c) western blot analysis and immunostaining of injected muscles.

Off target cleavage can be assessed in the five regions with closest sequence similarity to the target sites by deep sequencing. 5×10¹⁰ vector (vg) of the AAV6/CK8-nuclease and targeting vectors in a volume of 30 μl can be injected into eight TAs per time point (contralateral muscles can serve as the negative controls). The primary end points can be the percent positive myofibers in TAs, total dystrophin expression by western blot using previously described N- and C-terminal antibodies (Bengtsson, N. E., et al. Nat. Comm., 8, 14454, doi:10.1038/ncomms14454 (2017)), assembly of the DGC by immunostaining with commercial antibodies or those supplied by Stan Froehner (Bengtsson, N. E., et al. Nat. Comm., 8, 14454, doi:10.1038/ncomms14454 (2017)), and percent corrected dystrophin gene and mRNA. DGC expression can focus on nNOS and representative DGC members, β-dystroglycan, β-sarcoglycan, α1-syntrophin, and α-dystrobrevin-2. The goal can be to determine the relative efficiency of SpCas9, SaCas9, and the newer SpCas9-HF1 in muscle.

Systemic delivery studies can be performed using retro-orbital (RO) injection into 2-month-old mdx^{4cv} mice. Here, two enzymes can be compared, the HF1 enzyme and either the Sp or SaCas9, depending on which works best by IM. Methods can be as discussed above, at a moderate dose of 2×10¹² vg/25 g mouse weight of each vector, N=8 mice. Although the smaller SaCas9 and the sgRNAs fit into a single vector they can be split into two vectors as with SpCas9 and SpCas9-HF to maintain a constant vector particle number and for varying the ratios of the components. This dose is below the maximal gene delivery using conventional AAV vectors (such as microdystrophin), but as a non-saturating dose it can facilitate identifying efficiency differences. Analysis can be on skeletal muscles (e.g., TA, gastrocnemius, soleus, and diaphragm), the heart, and in select non-muscle tissues. Time points can be two and four months post-injection. As above, these comparisons can use CK8 to drive Cas9 expression. Endpoints can include dystrophin and DGC expression, genomic DNA targeting efficiencies, and off-target editing at the 5 regions closest in sequence to the sgRNA sequences.

Off target effects in non-muscle tissues may be undetectable due to the CK8 RC, but liver and kidney may be analyzed, which are targeted well by most AAV serotypes, including AAV6 (Gregorevic, P., et al. Nature Med. 10, 828-834, doi: 10.1038/nm 1085 (2004)).

Gene editing in the original mdx mouse has been conducted using the CMV promoter (see Tabebordbar, M., et al. Science, doi:10.1126/science.aad5177 (2015); Nelson, C. E., et al. Science, doi:10.1126/science.aad5143 (2015); and Long, C., et al. Science, doi:10.1126/science.aad5725 (2015)). CMV is active in non-muscle and immune effector cells, and was used in a clinical trial that led to a dystrophin immune response (Mendell J. R. et al. N. Engl. J. Med., 363, 1429-1437 (2010)). CK RCs have been optimized for high-level expression (20-80% of CMV) in various striated muscles (see Hauser M. A., et al. Mol. Ther. 2, 16-25 (2000); Salva, M. Z., et al. Mol. Ther. 15, 320-329 (2007); Himeda, C. L., et al. Methods Mol. Biol. 709, 3-19 (2011)). However, there could be advantages in using weaker promoters for Cas9. Some studies have been performed using the relatively weak EFS promoter, but poor editing efficiencies were seen (not shown). Here, a two-month time point (N=8 mice) can be used using the weaker CK6 RC and dystrophin expression and editing can be monitored in the muscles discussed above (Hauser, 2000). The simplest strategy of deleting exons 52-53 can be tested.

Combining Cas9 and sgRNAs into a single vector can be convenient but it locks in a ratio of enzyme to sgRNAs that may not be optimal. Using two vectors can allow one to vary the ratios of the components. The vector pairs can be injected RO into 12-week mdx^{4cv} mice using the most efficient Cas9 enzyme from the studies above (N=8/group). A constant total vector dose can be used (up to 4×10¹³ vg/25 g mouse), but 5 ratios may be tested, Cas9:sgRNA vector at 1:9; 2.5:7.5, 5:5, 7.5:2.5, and 9:1. 8 weeks post-injection, dystrophin and editing efficiencies (as above) can be analyzed. If the ratio of Cas9 to sgRNA proves important, the idea of testing regulatory cassettes with stronger/weaker activity to enable adjusting the ratios within a single vector can be revisited.

The optimal ratio of vectors can then be used for systemic delivery in a dose escalation study. Vector doses of 4, 8, and 12×10¹² vg/25 gram mouse weight, which is approaching the upper limit of delivery due to titer, volume and vector solubility concerns can be tested. N=8 mice/dose, and editing efficiencies, dystrophin, and DGC expression can be measured at 8 and 16-weeks post-injection.

The above studies can use 12-week-old (young adult) mice so as to impact the dystrophic phenotype in an already dystrophic animal. Also, mdx and mdx^{4cv} mice display an unusual wave of necrosis and regeneration from ˜4-10 weeks of age, a feature not shared by patients. This necrosis leads to significant loss of AAV vectors before gene expression peaks, since AAV vectors don't display optimal gene expression for ˜4 weeks, (see, e.g., Blankinship, 2004). However, gene editing efficiencies may be higher in younger mice than in adults. Therefore, at least one test can be performed in two-week old mice to compare with the results in 12-week mice. Here, the optimal parameters from the above studies can be tested by vector infusion into 2-week-old mdx^{4cv} mice (N=8), with analysis of editing and dystrophic production conducted 8 weeks later. Preliminary studies used mice at both 2 and 8-12 weeks of age and obvious differences were not observed, but those studies were pilot in nature and not well powered. Using an N=8 should provide sufficient statistical data to compare with the studies described above.

An extensive set of morphological and functional assays of dystrophic muscle function, including muscle and myofiber cross sectional area, central nucleation, blood chemistries, specific force and susceptibility to contraction-induced injury (in TA, gastrocnemius, EDL, soleus, and diaphragm); hemodynamic assays of cardiac function, and whole animal assays such as treadmill running, fatigue, gait, and hind-limb force have been published (see, e.g., Gregorevic, P., et al. Nature Med. 10, 828-834, doi:10.1038/nm1085 (2004); Gregorevic P. et al., Nature Med. 12, 787-789 (2006); Odom G et al, Mol. Ther., 16, 1539-1545 (2008), PMC2643133.

In these assays, the optimized (from A-D) CRISPR/Cas9 vectors can be injected retro-orbitally into 12-week old mdx^{4cv} mice (N=8/group) and analyzed for genomic and mRNA editing (by deep sequencing), dystrophin expression and pathophysiology in TA, gastrocnemius, soleus, diaphragm, and cardiac muscles at 3, 6, 12, and 24 months. Live animal assays can include cardiac Echo, hind-limb strength, fatigue, and gait. Mice may be analyzed in a blinded fashion. Other than breeders, the studies can use male mice as DMD affects males.

It will be apparent to those having skill in the art that many changes may be made to the details of the above-described embodiments without departing from the underlying principles of the invention. The scope of the present invention should, therefore, be determined only by the following claims.

1. A pharmaceutical composition comprising:
a muscle-specific nuclease cassette;
one or more guide RNA (gRNA) cassettes; and
a delivery system for delivery of the muscle-specific nuclease cassette and the one or more gRNA cassettes.

2. The pharmaceutical composition of claim 1, wherein the muscle-specific nuclease cassette comprises:
a muscle-specific transcriptional regulatory cassette; and
a nuclease coding sequence.

3. The pharmaceutical composition of claim 2, wherein the nuclease coding sequence encodes a CRISPR-associated nuclease.

4. The pharmaceutical composition of claim 2, wherein the muscle-specific transcriptional regulatory cassette is derived from at least one of an M-creatine kinase enhancer and an M-creatine kinase promoter.

5. The pharmaceutical composition of claim 1, further comprising a mutation-corrected DNA template, wherein the mutation-corrected DNA template is configured for homology directed repair.

6. The pharmaceutical composition of claim 5, wherein the mutation-corrected DNA template is configured to repair a mutated target nucleic acid sequence.

7. The pharmaceutical composition of claim 6, wherein the mutated target nucleic acid sequence is in a gene associated with a neuromuscular disorder.

8. The pharmaceutical composition of claim 6, wherein the mutated target nucleic acid sequence is in a gene encoding dystrophin.

9. The pharmaceutical composition of claim 1, wherein the delivery system comprises a recombinant adeno-associated virus (rAAV) vector.

10. The pharmaceutical composition of claim 9, wherein the rAAV vector is selected from at least one of an rAAV6 vector, an rAAV8 vector, and an rAAV9 vector.

11. The pharmaceutical composition of claim 1, wherein the delivery system comprises a first recombinant adeno-associated virus (rAAV) vector to deliver the muscle-specific nuclease cassette and a second rAAV vector to deliver the one or more g RNA cassettes.

12. The pharmaceutical composition of claim 11, wherein the first and second rAAV vectors are selected from at least one of an rAAV6 vector, an rAAV8 vector, and an rAAV9 vector.

13. The pharmaceutical composition of claim 1, wherein the pharmaceutical composition reduces a pathological effect or symptom of a neuromuscular disorder in a subject.

14. The pharmaceutical composition of claim 1, wherein the pharmaceutical composition increases a specific-force generating capacity of at least one skeletal muscle in the subject to within at least 40% of a normal specific-force generating capacity.

15. The pharmaceutical composition of claim 1, wherein the pharmaceutical composition restores a baseline end-diastolic volume defect in the subject to within at least 40% of a normal end-diastolic volume.

16. The pharmaceutical composition of claim 13, wherein the neuromuscular disorder is a muscular dystrophy selected from at least one of the myotonic muscular dystrophies (DM1 or DM2), Duchenne muscular dystrophy, Becker muscular dystrophy, the limb-girdle muscular dystrophies, the facioscapulohumeral muscular dystrophies, the congenital muscular dystrophies, oculopharyngeal muscular dystrophy, distal muscular dystrophy, the desmin-related myopathies, fukyama muscular dystrophy, the FKRP-deficiencies, and Emery-Dreifuss muscular dystrophy.

17. A method for treating a subject having a neuromuscular disorder, the method comprising:
administering to the subject a therapeutically effective amount of a pharmaceutical composition comprising:
a muscle-specific nuclease cassette;
one or more guide RNA (gRNA) cassettes; and
a delivery system for delivery of the muscle-specific nuclease cassette and the first gRNA cassette.

18. The method of claim 17, wherein the neuromuscular disorder is a muscular dystrophy selected from at least one of the myotonic muscular dystrophies (DM1 or DM2), Duchenne muscular dystrophy, Becker muscular dystrophy, the limb-girdle muscular dystrophies, the facioscapulohumeral muscular dystrophies, the congenital muscular dystrophies, oculopharyngeal muscular dystrophy, distal muscular dystrophy, the desmin-related myopathies, fukyama muscular dystrophy, the FKRP-deficiencies, and Emery-Dreifuss muscular dystrophy.

19. A method of modifying the sequence of a target nucleic acid sequence in a muscle cell, the method comprising:
transducing the muscle cell with a delivery system, the delivery system comprising:
a muscle-specific nuclease cassette; and
one or more guide RNA (gRNA) cassettes; and
a mutation-corrected DNA template comprising a modification to be made in the target nucleic acid sequence.

20. The method of claim 19, wherein the method further comprises inducing or reducing expression of a gene associated with a neuromuscular disorder in the muscle cell.


SEQUENCE LISTING (159 sequences)

SEQ ID NO: 1 - DNA, 20 bp, Artificial Sequence
  Synthetic oligonucleotide
gatactagggtggcaaatag

SEQ ID NO: 2 - DNA, 20 bp, Artificial Sequence
  Synthetic oligonucleotide
gtgttcttaaaagaatggtg

SEQ ID NO: 3 - DNA, 21 bp, Artificial Sequence
  Synthetic oligonucleotide
gatactagggtggcaaataga

SEQ ID NO: 4 - DNA, 22 bp, Artificial Sequence
  Synthetic oligonucleotide
gagataaatccctgcttatcac

SEQ ID NO: 5 - DNA, 20 bp, Artificial Sequence
  Synthetic oligonucleotide
gtcaagaacagctgcagaac

SEQ ID NO: 6 - DNA, 20 bp, Artificial Sequence
  Synthetic oligonucleotide
gcagttgaatgaaatgttaa

SEQ ID NO: 7 - DNA, 60 bp, Artificial Sequence
  Synthetic oligonucleotide
caagaacagctgcagaacaggagataacagttgaatgaaatgttaaaggattcaacacaa

SEQ ID NO: 8 - DNA, 60 bp, Mus musculus
caagaacagctgcagaacaggagacaacagttgaatgaaatgttaaaggattcaacacaa

SEQ ID NO: 9 - DNA, 60 bp, Artificial Sequence
  Synthetic oligonucleotide
caagaacagctgcagaacagaagacaacagttgaatgaaatgttaaaagattcaacacaa

SEQ ID NO: 10 - DNA, 60 bp, Artificial Sequence
  Synthetic oligonucleotide
caagaacagctgcagaacagaagacaacagttgaatgaaatgttaaaggattcaacacaa

SEQ ID NO: 11 - DNA, 60 bp, Artificial Sequence
  Synthetic oligonucleotide
caagaacagctgcagaacaggagacaacagttgaatgaaatgttaaaagattcaacacaa

SEQ ID NO: 12 - DNA, 60 bp, Artificial Sequence
  Synthetic oligonucleotide
caagaacagctgcagaacaggagagaacagttgaatgaaatgttaaaggattcaacacaa

SEQ ID NO: 13 - DNA, 60 bp, Artificial Sequence
  Synthetic oligonucleotide
caagaacagctgcagaacaggagaaaacagttgaatgaaatgttaaaggattcaacacaa

SEQ ID NO: 14 - DNA, 60 bp, Artificial Sequence
  Synthetic oligonucleotide
caagaacagctgcagaacagcagataacagttgaatgaaatgttaaaggattcaacacaa

SEQ ID NO: 15 - DNA, 60 bp, Artificial Sequence
  Synthetic oligonucleotide
caagaacagctgcagaacagtagataacagttgaatgaaatgttaaaggattcaacacaa

SEQ ID NO: 16 - DNA, 60 bp, Artificial Sequence
  Synthetic oligonucleotide
caagaacagctgcagaacaggagataacagttgaatgaaatgttaaacgattcaacacaa

SEQ ID NO: 17 - DNA, 60 bp, Artificial Sequence
  Synthetic oligonucleotide
caagaacagctgcagaacaggagataacagttgaatgaaatgttaaatgattcaacacaa

SEQ ID NO: 18 - DNA, 60 bp, Artificial Sequence; identical to SEQ ID NO: 7
  Synthetic oligonucleotide

SEQ ID NO: 19 - DNA, 57 bp, Artificial Sequence
  Synthetic oligonucleotide
caagaacagctgcagaacaggagacagttgaatgaaatgttaaaggattcaacacaa

SEQ ID NO: 20 - DNA, 54 bp, Artificial Sequence
  Synthetic oligonucleotide
caagaacagctgcagaacaggcagttgaatgaaatgttaaaggattcaacacaa

SEQ ID NO: 21 - DNA, 51 bp, Artificial Sequence
  Synthetic oligonucleotide
caagaacagctgcagaaccagttgaatgaaatgttaaaggattcaacacaa

SEQ ID NO: 22 - DNA, 48 bp, Artificial Sequence
  Synthetic oligonucleotide
caagaacagctgcagcagttgaatgaaatgttaaaggattcaacacaa

SEQ ID NO: 23 - DNA, 45 bp, Artificial Sequence
  Synthetic oligonucleotide
caagaacagctgcagaacagggaaatgttaaaggattcaacacaa

SEQ ID NO: 24 - DNA, 45 bp, Artificial Sequence
  Synthetic oligonucleotide
caagaacagctgcagaacaggagaatgttaaaggattcaacacaa

SEQ ID NO: 25 - DNA, 42 bp, Artificial Sequence
  Synthetic oligonucleotide
caagaacagctgcagaacaggatgttaaaggattcaacacaa

SEQ ID NO: 26 - DNA, 42 bp, Artificial Sequence
  Synthetic oligonucleotide
caagaacagctgcagaacaggagattaaaggattcaacacaa

SEQ ID NO: 27 - DNA, 39 bp, Artificial Sequence
  Synthetic oligonucleotide
caagaacagctgcagaacatgttaaaggattcaacacaa

SEQ ID NO: 28 - DNA, 39 bp, Artificial Sequence
  Synthetic oligonucleotide
caagaacagctgcagaacaggttaaaggattcaacacaa

SEQ ID NO: 29 - DNA, 36 bp, Artificial Sequence
  Synthetic oligonucleotide
caagaacagctgcagatgttaaaggattcaacacaa

SEQ ID NO: 30 - DNA, 36 bp, Artificial Sequence
  Synthetic oligonucleotide
caagaacagctgcagaacttaaaggattcaacacaa

SEQ ID NO: 31 - DNA, 33 bp, Artificial Sequence
  Synthetic oligonucleotide
caagaacagctgcagaacaaggattcaacacaa

SEQ ID NO: 32 - DNA, 33 bp, Artificial Sequence
  Synthetic oligonucleotide
caagaacagctgcagttaaaggattcaacacaa

SEQ ID NO: 33 - DNA, 30 bp, Artificial Sequence
  Synthetic oligonucleotide
caagaacagctgttaaaggattcaacacaa

SEQ ID NO: 34 - DNA, 30 bp, Artificial Sequence
  Synthetic oligonucleotide
caagaacagctgcagaaggattcaacacaa

SEQ ID NO: 35 - DNA, 27 bp, Artificial Sequence
  Synthetic oligonucleotide
caagaacagctgcaggattcaacacaa

SEQ ID NO: 36 - DNA, 27 bp, Artificial Sequence
  Synthetic oligonucleotide
caagaacagctgaaggattcaacacaa

SEQ ID NO: 37 - DNA, 24 bp, Artificial Sequence
  Synthetic oligonucleotide
caagaacagaaggattcaacacaa

SEQ ID NO: 38 - DNA, 24 bp, Artificial Sequence
  Synthetic oligonucleotide
caagaacagctggattcaacacaa

SEQ ID NO: 39 - DNA, 23 bp, Artificial Sequence
  Synthetic oligonucleotide
cagctgcagtaaaggattcaaca

SEQ ID NO: 40 - DNA, 91 bp, Artificial Sequence
  Synthetic oligonucleotide
atcgaattgaaagaattcagattcagtgggatgaggttcaagaacagctgcagaacaggagataacagttgaatgaaatgttaaaggattc

SEQ ID NO: 41 - DNA, 91 bp, Mus musculus
atcgaattgaaagaattcagattcagtgggatgaggttcaagaacagctgcagaacaggagacaacagttgaatgaaatgttaaaggattc

SEQ ID NO: 42 - DNA, 91 bp, Artificial Sequence
  Synthetic oligonucleotide
atcgaattgaaagaattcagattcagtgggatgaggttcaagaacagctgcagaacagaagacaacagttgaatgaaatgttaaaagattc

SEQ ID NO: 43 - DNA, 91 bp, Artificial Sequence
  Synthetic oligonucleotide
atcgaattgaaagaattcagattcagtgggatgaggttcaagaacagctgcagaacagaagacaacagttgaatgaaatgttaaaggattc

SEQ ID NO: 44 - DNA, 91 bp, Artificial Sequence
  Synthetic oligonucleotide
atcgaattgaaagaattcagattcagtgggatgaggttcaagaacagctgcagaacaggagacaacagttgaatgaaatgttaaaagattc

SEQ ID NO: 45 - DNA, 91 bp, Artificial Sequence
  Synthetic oligonucleotide
atcgaattgaaagaattcagattcagtgggatgaggttcaagaacagctgcagaacaggagagaacagttgaatgaaatgttaaaggattc

SEQ ID NO: 46 - DNA, 91 bp, Artificial Sequence
  Synthetic oligonucleotide
atcgaattgaaagaattcagattcagtgggatgaggttcaagaacagctgcagaacaggagaaaacagttgaatgaaatgttaaaggattc

SEQ ID NO: 47 - DNA, 91 bp, Artificial Sequence
  Synthetic oligonucleotide
atcgaattgaaagaattcagattcagtgggatgaggttcaagaacagctgcagaacagcagataacagttgaatgaaatgttaaaggattc

SEQ ID NO: 48 - DNA, 91 bp, Artificial Sequence
  Synthetic oligonucleotide
atcgaattgaaagaattcagattcagtgggatgaggttcaagaacagctgcagaacagtagataacagttgaatgaaatgttaaaggattc

SEQ ID NO: 49 - DNA, 91 bp, Artificial Sequence
  Synthetic oligonucleotide
atcgaattgaaagaattcagattcagtgggatgaggttcaagaacagctgcagaacaggagataacagttgaatgaaatgttaaacgattc

SEQ ID NO: 50 - DNA, 91 bp, Artificial Sequence
  Synthetic oligonucleotide
atcgaattgaaagaattcagattcagtgggatgaggttcaagaacagctgcagaacaggagataacagttgaatgaaatgttaaatgattc

SEQ ID NO: 51 - DNA, 91 bp, Artificial Sequence; identical to SEQ ID NO: 40
  Synthetic oligonucleotide

SEQ ID NO: 52 - DNA, 88 bp, Artificial Sequence
  Synthetic oligonucleotide
atcgaattgaaagaattcagattcagtgggatgaggttcaagaacagctgcagaacaggagacagttgaatgaaatgttaaaggattc

SEQ ID NO: 53 - DNA, 85 bp, Artificial Sequence
  Synthetic oligonucleotide
atcgaattgaaagaattcagattcagtgggatgaggttcaagaacagctgcagaacaggcagttgaatgaaatgttaaaggattc

SEQ ID NO: 54 - DNA, 82 bp, Artificial Sequence
  Synthetic oligonucleotide
atcgaattgaaagaattcagattcagtgggatgaggttcaagaacagctgcagaaccagttgaatgaaatgttaaaggattc

SEQ ID NO: 55 - DNA, 79 bp, Artificial Sequence
  Synthetic oligonucleotide
atcgaattgaaagaattcagattcagtgggatgaggttcaagaacagctgcagcagttgaatgaaatgttaaaggattc

SEQ ID NO: 56 - DNA, 76 bp, Artificial Sequence
  Synthetic oligonucleotide
atcgaattgaaagaattcagattcagtgggatgaggttcaagaacagctgcagaacagggaaatgttaaaggattc

SEQ ID NO: 57 - DNA, 76 bp, Artificial Sequence
  Synthetic oligonucleotide
atcgaattgaaagaattcagattcagtgggatgaggttcaagaacagctgcagaacaggagaatgttaaaggattc

SEQ ID NO: 58 - DNA, 73 bp, Artificial Sequence
  Synthetic oligonucleotide
atcgaattgaaagaattcagattcagtgggatgaggttcaagaacagctgcagaacaggatgttaaaggattc

SEQ ID NO: 59 - DNA, 73 bp, Artificial Sequence
  Synthetic oligonucleotide
atcgaattgaaagaattcagattcagtgggatgaggttcaagaacagctgcagaacaggagattaaaggattc

SEQ ID NO: 60 - DNA, 70 bp, Artificial Sequence
  Synthetic oligonucleotide
atcgaattgaaagaattcagattcagtgggatgaggttcaagaacagctgcagaacatgttaaaggattc

SEQ ID NO: 61 - DNA, 70 bp, Artificial Sequence
  Synthetic oligonucleotide
atcgaattgaaagaattcagattcagtgggatgaggttcaagaacagctgcagaacaggttaaaggattc

SEQ ID NO: 62 - DNA, 67 bp, Artificial Sequence
  Synthetic oligonucleotide
atcgaattgaaagaattcagattcagtgggatgaggttcaagaacagctgcagatgttaaaggattc

SEQ ID NO: 63 - DNA, 67 bp, Artificial Sequence
  Synthetic oligonucleotide
atcgaattgaaagaattcagattcagtgggatgaggttcaagaacagctgcagaacttaaaggattc

SEQ ID NO: 64 - DNA, 64 bp, Artificial Sequence
  Synthetic oligonucleotide
atcgaattgaaagaattcagattcagtgggatgaggttcaagaacagctgcagaacaaggattc

SEQ ID NO: 65 - DNA, 64 bp, Artificial Sequence
  Synthetic oligonucleotide
atcgaattgaaagaattcagattcagtgggatgaggttcaagaacagctgcagttaaaggattc

SEQ ID NO: 66 - DNA, 61 bp, Artificial Sequence
  Synthetic oligonucleotide
atcgaattgaaagaattcagattcagtgggatgaggttcaagaacagctgttaaaggattc

SEQ ID NO: 67 - DNA, 61 bp, Artificial Sequence
  Synthetic oligonucleotide
atcgaattgaaagaattcagattcagtgggatgaggttcaagaacagctgcagaaggattc

SEQ ID NO: 68 - DNA, 58 bp, Artificial Sequence
  Synthetic oligonucleotide
atcgaattgaaagaattcagattcagtgggatgaggttcaagaacagctgcaggattc

SEQ ID NO: 69 - DNA, 58 bp, Artificial Sequence
  Synthetic oligonucleotide
atcgaattgaaagaattcagattcagtgggatgaggttcaagaacagctgaaggattc

SEQ ID NO: 70 - DNA, 55 bp, Artificial Sequence
  Synthetic oligonucleotide
atcgaattgaaagaattcagattcagtgggatgaggttcaagaacagaaggattc

SEQ ID NO: 71 - DNA, 55 bp, Artificial Sequence
  Synthetic oligonucleotide
atcgaattgaaagaattcagattcagtgggatgaggttcaagaacagctggattc

SEQ ID NO: 72 - DNA, 63 bp, Artificial Sequence
  Synthetic oligonucleotide
atcgaattgaaagaattcagattcagtgggatgaggttcaagaacagctgcagtaaaggattc

SEQ ID NO: 73 - DNA, 20 bp, Artificial Sequence
  Synthetic oligonucleotide
gatactagtgtggctcatag

SEQ ID NO: 74 - DNA, 20 bp, Artificial Sequence
  Synthetic oligonucleotide
gatacgatggtggcaaatcg

SEQ ID NO: 75 - DNA, 20 bp, Artificial Sequence
  Synthetic oligonucleotide
gatactagggtggggaataa

SEQ ID NO: 76 - DNA, 20 bp, Artificial Sequence
  Synthetic oligonucleotide
tttttcttaaaagaatggta

SEQ ID NO: 77 - DNA, 20 bp, Artificial Sequence
  Synthetic oligonucleotide
ttgatcttagaagaatggtg

SEQ ID NO: 78 - DNA, 20 bp, Artificial Sequence
  Synthetic oligonucleotide
gttttcttgaaaaaatggtg

SEQ ID NO: 79 - DNA, 20 bp, Artificial Sequence
  Synthetic oligonucleotide
ctgttcttaaaaggttggtg

SEQ ID NO: 80 - DNA, 20 bp, Artificial Sequence
  Synthetic oligonucleotide
gagttcttcaaagaatagtg

SEQ ID NO: 81 - DNA, 19 bp, Artificial Sequence
  Synthetic oligonucleotide
tctagggcagctgcagaac

SEQ ID NO: 82 - DNA, 19 bp, Artificial Sequence
  Synthetic oligonucleotide
tcattcacagctgcagaac

SEQ ID NO: 83 - DNA, 19 bp, Artificial Sequence
  Synthetic oligonucleotide
caaagaatagctgcagaac

SEQ ID NO: 84 - DNA, 19 bp, Artificial Sequence
  Synthetic oligonucleotide
tcaagaacagctgcagcag

SEQ ID NO: 85 - DNA, 19 bp, Artificial Sequence
  Synthetic oligonucleotide
tcaagaacagctgcatcac

SEQ ID NO: 86 - DNA, 19 bp, Artificial Sequence
  Synthetic oligonucleotide
cagttacatgaaatgttaa

SEQ ID NO: 87 - DNA, 19 bp, Artificial Sequence
  Synthetic oligonucleotide
cattttaatgaaatgttaa

SEQ ID NO: 88 - DNA, 19 bp, Artificial Sequence
  Synthetic oligonucleotide
aagttgaatgaaattttaa

SEQ ID NO: 89 - DNA, 19 bp, Artificial Sequence
  Synthetic oligonucleotide
cagtggaataaaatgttaa

SEQ ID NO: 90 - DNA, 808 bp, Artificial Sequence
  Synthetic oligonucleotide
gtacctttctaataaataattgttatttagtgtcagagtctaaagttgaatttatatttctaaacatggcaccaatattgtagtttatttcaatgcaagtaatttaatagaaagtcaaatttgtcacctgaagaaatgattttgttaattattttacctatatcactcatagcaccttggatatatttaatgagaaatatacatgtgcaatgacgtttagattctaaatttccactgtcttctcttgagtaataattactgttctttattcttatttttattccagttgaaagaattcagattcagtgggatgaggttcaagaacagctgcagaacagaagacaacagttgaatgaaatgttaaaagattcaacacaatggctggaagctaaggaagaagccgaacaggtcataggacaggtcagaggcaagcttgactcatggaaagaaggtcctcacacagtagatgcaatccaaaagaagatcacagaaaccaaggttagtgtcaagcatatctttaaaaaaatattttgtatagcaaatgaaagcatgccataaattaaaatttaatgttttcttagtgaaaattacatttaggaagtgaaaagtggaattcttgcttgtttttgattggttggtttgttggttggttggttggttggctggctggttggttggttggctggttggttggttggttggttttgagacaaaaatctaaactcaaaatactcaagactacagatgagtgccactacatctacatgatttaaaattttgagacacagtataggttataggaaaactg

SEQ ID NO: 91 - DNA, 20 bp, Artificial Sequence
  Synthetic oligonucleotide
atggccccaaagaagaagcg

SEQ ID NO: 92 - DNA, 21 bp, Artificial Sequence
  Synthetic oligonucleotide
cttactttttcttttttgcct

SEQ ID NO: 93 - DNA, 18 bp, Artificial Sequence
  Synthetic oligonucleotide
tcagacccacctcccaac

SEQ ID NO: 94 - DNA, 22 bp, Artificial Sequence
  Synthetic oligonucleotide
aattcaaaaaagcaccgactcg

SEQ ID NO: 95 - DNA, 18 bp, Artificial Sequence
  Synthetic oligonucleotide
tcagacccacctcccaac

SEQ ID NO: 96 - DNA, 22 bp, Artificial Sequence
  Synthetic oligonucleotide
aattcaaaaaagcaccgactcg

SEQ ID NO: 97 - DNA, 24 bp, Artificial Sequence
  Synthetic oligonucleotide
caccgatactagggtggcaaatag

SEQ ID NO: 98 - DNA, 24 bp, Artificial Sequence
  Synthetic oligonucleotide
aaacctatttgccaccctagtatc

SEQ ID NO: 99 - DNA, 24 bp, Artificial Sequence
  Synthetic oligonucleotide
caccgtgttcttaaaagaatggtg

SEQ ID NO: 100 - DNA, 24 bp, Artificial Sequence
  Synthetic oligonucleotide
aaaccaccattcttttaagaacac

SEQ ID NO: 101 - DNA, 25 bp, Artificial Sequence
  Synthetic oligonucleotide
caccgatactagggtggcaaataga

SEQ ID NO: 102 - DNA, 25 bp, Artificial Sequence
  Synthetic oligonucleotide
aaactctatttgccaccctagtatc

SEQ ID NO: 103 - DNA, 26 bp, Artificial Sequence
  Synthetic oligonucleotide
caccgagataaatccctgcttatcac

SEQ ID NO: 104 - DNA, 26 bp, Artificial Sequence
  Synthetic oligonucleotide
aaacgtgataagcagggatttatctc

SEQ ID NO: 105 - DNA, 24 bp, Artificial Sequence
  Synthetic oligonucleotide
caccgtcaagaacagctgcagaac

SEQ ID NO: 106 - DNA, 24 bp, Artificial Sequence
  Synthetic oligonucleotide
aaacgttctgcagctgttcttgac

SEQ ID NO: 107 - DNA, 24 bp, Artificial Sequence
  Synthetic oligonucleotide
caccgcagttgaatgaaatgttaa

SEQ ID NO: 108 - DNA, 24 bp, Artificial Sequence
  Synthetic oligonucleotide
aaacttaacatttcattcaactgc

SEQ ID NO: 109 - DNA, 20 bp, Artificial Sequence
  Synthetic oligonucleotide
ctcatacccaaagctgctag

SEQ ID NO: 110 - DNA, 20 bp, Artificial Sequence
  Synthetic oligonucleotide
ccttttagcctagagagtgc

SEQ ID NO: 111 - DNA, 20 bp, Artificial Sequence
  Synthetic oligonucleotide
gctgaggtaatagagccaag

SEQ ID NO: 112 - DNA, 22 bp, Artificial Sequence
  Synthetic oligonucleotide
ctgtgatcttcttttggattgc

SEQ ID NO: 113 - DNA, 20 bp, Artificial Sequence
  Synthetic oligonucleotide
gccatcttctttgctgttgg

SEQ ID NO: 114 - DNA, 20 bp, Artificial Sequence
  Synthetic oligonucleotide
tcccgaagaagtttcagtgc

SEQ ID NO: 115 - DNA, 20 bp, Artificial Sequence
  Synthetic oligonucleotide
cagagagtgatggtgggtga

SEQ ID NO: 116 - DNA, 20 bp, Artificial Sequence
  Synthetic oligonucleotide
tcccgaagaagtttcagtgc

SEQ ID NO: 117 - DNA, 30 bp, Artificial Sequence
  Synthetic oligonucleotide
cacacatttgtccttatgattaagatttgg

SEQ ID NO: 118 - DNA, 24 bp, Artificial Sequence
  Synthetic oligonucleotide
gcaacaacactcttaaacactgag

SEQ ID NO: 119 - DNA, 22 bp, Artificial Sequence
  Synthetic oligonucleotide
atgtcctttgccaccatgctaa

SEQ ID NO: 120 - DNA, 29 bp, Artificial Sequence
  Synthetic oligonucleotide
gcatctattacttttcctaagaagaaatt

SEQ ID NO: 121 - DNA, 24 bp, Artificial Sequence
  Synthetic oligonucleotide
tttccactgtcttctcttgagtaa

SEQ ID NO: 122 - DNA, 23 bp, Artificial Sequence
  Synthetic oligonucleotide
ctactgtgtgaggaccttctttc

SEQ ID NO: 123 - DNA, 22 bp, Artificial Sequence
  Synthetic oligonucleotide
ccagcaatcaagaagctagaac

SEQ ID NO: 124 - DNA, 20 bp, Artificial Sequence
  Synthetic oligonucleotide
gcatctactgtgtgaggacc

SEQ ID NO: 125 - DNA, 19 bp, Artificial Sequence
  Synthetic oligonucleotide
ctgtttgctcatgggctca

SEQ ID NO: 126 - DNA, 23 bp, Artificial Sequence
  Synthetic oligonucleotide
acagtctttcataacagaatgct

SEQ ID NO: 127 - DNA, 20 bp, Artificial Sequence
  Synthetic oligonucleotide
ccaacgactgtttctgaacc

SEQ ID NO: 128 - DNA, 22 bp, Artificial Sequence
  Synthetic oligonucleotide
gaaatgtctctacccagtttgc

SEQ ID NO: 129 - DNA, 23 bp, Artificial Sequence
  Synthetic oligonucleotide
gccatcctcaaagaagaaccaag

SEQ ID NO: 130 - DNA, 22 bp, Artificial Sequence
  Synthetic oligonucleotide
tggagcaccacatttagccatc

SEQ ID NO: 131 - DNA, 22 bp, Artificial Sequence
  Synthetic oligonucleotide
gatggctttgaacttgagagag

SEQ ID NO: 132 - DNA, 20 bp, Artificial Sequence
  Synthetic oligonucleotide
ctaacacactcggggaactg

SEQ ID NO: 133 - DNA, 22 bp, Artificial Sequence
  Synthetic oligonucleotide
gagttgattctccttccacatc

SEQ ID NO: 134 - DNA, 20 bp, Artificial Sequence
  Synthetic oligonucleotide
ttcgttccacctaccagaag

SEQ ID NO: 135 - DNA, 19 bp, Artificial Sequence
  Synthetic oligonucleotide
agccttagcgttggtacag

SEQ ID NO: 136 - DNA, 26 bp, Artificial Sequence
  Synthetic oligonucleotide
attcattacctcatagttatgctgct

SEQ ID NO: 137 - DNA, 24 bp, Artificial Sequence
  Synthetic oligonucleotide
cctgtaaccagacaagacttctag

SEQ ID NO: 138 - DNA, 24 bp, Artificial Sequence
  Synthetic oligonucleotide
gtcactggtagttaatagggtacg

SEQ ID NO: 139 - DNA, 22 bp, Artificial Sequence
  Synthetic oligonucleotide
cacctgttgacgacagtaacaa

SEQ ID NO: 140 - DNA, 25 bp, Artificial Sequence
  Synthetic oligonucleotide
agtcaaatgacaagactatggctac

SEQ ID NO: 141 - DNA, 20 bp, Artificial Sequence
  Synthetic oligonucleotide
cgagccacagtgaattgatg

SEQ ID NO: 142 - DNA, 21 bp, Artificial Sequence
  Synthetic oligonucleotide
cgttccatagacaagtgtcag

SEQ ID NO: 143 - DNA, 19 bp, Artificial Sequence
  Synthetic oligonucleotide
gctgcctgggaggtttaga

SEQ ID NO: 144 - DNA, 19 bp, Artificial Sequence
  Synthetic oligonucleotide
ggttcagcctctcagtggg

SEQ ID NO: 145 - DNA, 21 bp, Artificial Sequence
  Synthetic oligonucleotide
caatggagcagaaaacgtctg

SEQ ID NO: 146 - DNA, 21 bp, Artificial Sequence
  Synthetic oligonucleotide
gcacagtccacatcctcattc

SEQ ID NO: 147 - DNA, 22 bp, Artificial Sequence
  Synthetic oligonucleotide
ggcttggttcacttttccactc

SEQ ID NO: 148 - DNA, 22 bp, Artificial Sequence
  Synthetic oligonucleotide
ctcagtggcagttgaataccag

SEQ ID NO: 149 - DNA, 23 bp, Artificial Sequence
  Synthetic oligonucleotide
gtctcagtctcagtgaacaagtg

SEQ ID NO: 150 - DNA, 21 bp, Artificial Sequence
  Synthetic oligonucleotide
ggcacagcatgtactagagat

SEQ ID NO: 151 - DNA, 18 bp, Artificial Sequence
  Synthetic oligonucleotide
agcagggttcgcttgtag

SEQ ID NO: 152 - DNA, 20 bp, Artificial Sequence
  Synthetic oligonucleotide
gctcacttgactttgaaggc

SEQ ID NO: 153 - DNA, 20 bp, Artificial Sequence
  Synthetic oligonucleotide
agcatggacaatgaggaact

SEQ ID NO: 154 - DNA, 20 bp, Artificial Sequence
  Synthetic oligonucleotide
cctgggtgttttgcttttct

SEQ ID NO: 155 - DNA, 22 bp, Artificial Sequence
  Synthetic oligonucleotide
tacaggtgagttgacacactcc

SEQ ID NO: 156 - DNA, 24 bp, Artificial Sequence
  Synthetic oligonucleotide
aggatttcagcatcactatctagc

SEQ ID NO: 157 - DNA, 21 bp, Artificial Sequence
  Synthetic oligonucleotide
ccttgccattctgccttttcc

SEQ ID NO: 158 - DNA, 22 bp, Artificial Sequence
  Synthetic oligonucleotide
cattctgtgtgaggtaacccag

SEQ ID NO: 159 - DNA, 450 bp, Artificial Sequence
  Synthetic oligonucleotide
ctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc